(19) United States
(12) Patent Application Publication
(10) Pub. No.: US 2014/0178884 A1
(43) Pub. Date: Jun. 26, 2014

(54) METHOD OF DIAGNOSING AND TREATING AN ASPERGILLUS SPECIES-ASSOCIATED CONDITION

(71) Applicants: David Aucoin, Reno, NV (US); Thomas R. Kozel, Reno, NV (US); Sindy Chaves, Reno, NV (US)

(72) Inventors: David Aucoin, Reno, NV (US); Thomas R. Kozel, Reno, NV (US); Sindy Chaves, Reno, NV (US)

(73) Assignee: BOARD OF REGENTS OF THE NEVADA SYSTEM OF HIGHER EDUCATION, ON BEHALF OF THE UNIV. OF NEVADA, RENO, Reno, NV (US)

(21) Appl. No.: 14/118,502

(22) PCT Filed: May 22, 2012
(86) PCT No.: PCT/US2012/039012

§ 371 Date: Dec. 27, 2013

Related U.S. Application Data
(60) Provisional application No. 61/489,180, filed on May 23, 2011.
(60) Provisional application No. 61/565,401, filed on Nov. 30, 2011.

(51) Int. Cl. G01N 33/569

(52) U.S. Cl. CPC G01N 33/56961
USPC 435/6.12; 436/501; 435/7.92; 435/7.1; 435/7.4; 435/7.31; 435/7.94; 435/7.93

(57) ABSTRACT

Disclosed herein are methods of diagnosing, monitoring and treating an Aspergillus species-associated condition, such as Aspergillus fumigatus (Af)-associated condition, including aspergillosis, such as invasive aspergillosis (IA). Methods for diagnosing and/or monitoring an Aspergillus species-associated condition, such as IA are provided. Also disclosed are point-of-care immunoassays that can be used to diagnose or monitor the efficacy of an Aspergillus-associated condition treatment. These immunoassays can also be used for rapid diagnosis of infection produced by an Aspergillus species, such as Af.

### CROSS REFERENCE TO RELATED APPLICATIONS

This application claims the benefit of U.S. Provisional Application No. 61/489,180, filed May 23, 2011, and U.S. Provisional Application No. 61/565,401, filed Nov. 30, 2011, each of which is incorporated by reference herein in its entirety.

### ACKNOWLEDGMENT OF GOVERNMENT SUPPORT

This invention was made with United States government support pursuant to Grant No. AI085548 from the National Institutes of Health/National Institute of Allergy and Infectious Diseases and Grant No. P20RR-016464 from the IDeA Networks of Biomedical Research Excellence (INBRE) Program of the National Center for Research Resources of the National Institutes of Health. The United States Government has certain rights in the invention.

### FIELD

This relates to the field of Aspergillus species and specifically to the identification of subjects who have an Aspergillus species-associated condition, such as invasive aspergillosis, by detecting Aspergillus species specific antigens, such as Aspergillus fumigatus specific antigens and monitoring the effectiveness of treatments for such conditions.

### BACKGROUND

Invasive aspergillosis (IA) is a serious opportunistic fungal infection caused mainly by Aspergillus fumigatus (Af). Symptoms of IA are often non-specific, making the disease difficult to diagnose. Bone marrow and solid organ transplant recipients, cancer patients undergoing chemotherapy and patients with chronic granulomatous disease are particularly at risk of developing IA. This disease is associated with high mortality rates because the symptoms are often non-specific, making it difficult to obtain an accurate diagnosis at the onset of the disease. Early diagnosis is critical for treatment to be effective. Unfortunately, diagnostic tools are not only limited but diagnosis with current methods often occurs when the fungal burden is too high for treatment to be efficient.

### SUMMARY

A point-of-care immunoassay for diagnosis of an Aspergillus species-associated condition, such as an Af-associated condition, including agents capable of detecting low levels of an Aspergillus species, such as low levels of Af, could greatly impact patient outcome because they would be able to detect the Aspergillus species, such as Af, and thus a condition associated with an Aspergillus species, such as IA, within minutes or hours from testing as compared to days required with the current methods. Earlier detection translates into earlier administration of therapies which could significantly increase the likelihood of patient survival as well as decrease the severity of the disease. The critical issue relies on a successful strategy to identify fungal antigens that are shed into body fluids during infection. For this purpose, the inventors used In vivo Microbial Antigen Discovery (InMAD). The hypothesis behind this technique is that serum or urine from mice or guinea pigs infected with Aspergillus contains precisely those antigens that would be targets for immunoassay.

In the InMAD technique, immunosuppressed subjects were infected with Af spores. Serum and urine were collected and filtered to remove whole fungal cells but leave behind soluble antigens released during infection. The filtered samples were used to immunize naïve subjects and their serum was collected to identify antigens recognized by antibodies using 1D and 2D immunoblots prepared from fungal whole cell lysates. Mass spectroscopy was used to identify those reactive antigens.

The inventors identified Aspergillus species protein antigens, such as Af protein antigens, that are present in subjects with an Aspergillus species-associated condition, such as an Af-associated condition (see Table 1). As indicated in Table 1, InMAD detected fifteen Aspergillus species antigens that were shed into serum and urine during infection. Many of the proteins that were observed to be present in serum from the infected animals were also found in urine. Immunization was found to be achieved across host species demonstrating that the InMAD technique can identify secreted antigens in serum and urine from an Af animal model of infection. These studies support the use of the protein antigens identified by InMAD for diagnosing a condition associated with an Aspergillus species, such as an Af-associated condition, including IA. In particular, these results suggest that Catalase (Protein ID No. 70986104), GPI-anchored cell wall β-1,3-endogluconase (Eglc) (Protein ID No. 70985687), GPI-anchored cell wall organization protein (Ecm33) (Protein ID No. 70994734), Chr-like protein (Protein ID No. 27372089), Thioreduxin reductase (Protein ID No. 70992029), Peptidyl-prolyl cis-trans isomerase (Protein ID No. 70989309), Major allergen (Protein ID No. 83300352), Conserved hypothetical protein (Protein ID No. 159122886), Conserved hypothetical protein (Protein ID No. 70995516), 1,3-β-glucanosyltransferase (Protein ID No. 70989629), Adenoside deaminase (Protein ID No. 146323525), L-amino acid oxidase Lao (Protein ID No. 70986680), Glu/Leu/Phe/Val Dehydrogenase (Protein ID No. 70994774), Adenosyl homocysteinase (Protein ID No. 70995231), Pigment biosynthesis protein (Protein ID No. 211909651) are indicators of an Aspergillus species-associated condition, such as an Af-associated condition, including an aspergillosis infection, such as IA.

Based on these findings, disclosed herein are methods of diagnosing and monitoring an Aspergillus species-associated condition, such as an Af-associated condition including aspergillosis, such as IA. Methods for diagnosing and/or monitoring an Aspergillus species-associated condition, such as an Af-associated condition (e.g., IA) are provided. Also disclosed are point-of-care immunoassays that can be used to diagnose or monitor the efficacy of an Aspergillus species-associated condition treatment, such as an Af-associated condition treatment. These immunoassays can also be used for rapid diagnosis of infection produced by an Aspergillus species, such as Af. These immunoassays can also be used for self monitoring in which a subject, such as an immunosuppressed patient, monitors the presence of one or Aspergillus species, such as Af, to monitor the onset of an infection.

In one particular embodiment, a method of diagnosing a subject with an Aspergillus species-associated condition, including an Af-associated condition, such as IA, or monitoring the efficacy of a therapy comprises detecting at least one Aspergillus species-associated molecule, such as an Af-associated molecule alone or in combination in a sample obtained from a subject exhibiting one or more signs or symptoms associated with an Aspergillus species-associated condition, such as an Af-associated condition, including one or more signs or symptoms associated with IA or a subject known to be at risk of acquiring an Aspergillus species-associated condition, such as an Af-associated condition, thereby diagnosing the subject. In one example, at least one Aspergillus species-associated molecule is an Af-associated molecule, including at least one antigen listed in Table 1. In one example, the at least one antigen listed in Table 1 is at least one of Af Catalase, GPI-anchored cell wall β-1,3-endogluconase (Eglc), GPI-anchored cell wall organization protein (Ecm33), Chr-like protein, Thioreduxin reductase. In one example, detecting at least one Af-associated molecule includes detecting at least Af catalase and GPI-anchored cell wall beta 1,3-endoglucanase (Eglc), or a combination thereof.

In one example, detecting at least one Aspergillus species-associated molecule, includes detecting at least one of Aspergillus Catalase (Protein ID No. 70986104), GPI-anchored cell wall β-1,3-endogluconase (Eglc) (Protein ID No. 70985687), GPI-anchored cell wall organization protein (Ecm33) (Protein ID No. 70994734), Chr-like protein (Protein ID No. 27372089), Thioreduxin reductase (Protein ID No. 70992029), Peptidyl-prolyl cis-trans isomerase (Protein ID No. 70989309), Major allergen (83300352), Conserved hypothetical protein (Protein ID No. 159122886), Conserved hypothetical protein (Protein ID No. 70995516), 1,3-β-glucanosyltransferase (Protein ID No. 70989629), Adenoside deaminase (Protein ID No. 146323525), L-amino acid oxidase Lao (Protein ID No. 70986680), Glu/Leu/Phe/Val Dehydrogenase (Protein ID No. 70994774), Adenosyl homocysteinase (Protein ID No. 70995231), or Pigment biosynthesis protein (Protein ID No. 211909651).

In some examples, the method further comprises comparing detection of the at least one Aspergillus species-associated molecule, such as at least one Af-associated molecule, in the sample obtained from the subject exhibiting one or more signs or symptoms associated with an Aspergillus species-associated condition to a control, wherein increased detection of the at least one Aspergillus species-associated molecule, such as an Af-associated molecule, relative to a control indicates that the subject has an Aspergillus species-associated condition, such as Af-associated condition.

In one example, detecting of that at least one Aspergillus species-associated molecule, such as an Af-associated molecule, comprises usage of at least one antibody specific for the at least one Af-associated molecule, such as an antibody specific for one or more of the antigens present in Table 1.

In one example, detecting of the at least one Aspergillus species-associated molecule, such as an Af-associated molecule, comprises usage of at least one aptamer specific for the at least one Af-associated molecule, such as an aptamer specific for one or more molecules present in Table 1.

In some examples, the method is used for detecting any condition or disease associated with an Aspergillus species, such as Af, including IA. In some examples, the disclosed method is used for diagnosing a subject with IA. In some examples, the method is a method for monitoring the efficacy of therapy. In some examples, the method is a method for self-monitoring or frequent monitoring for onset of an Aspergillus species-associated condition,

In one example, detecting at least one Af-associated molecule comprises using a lateral flow device.

In some examples, the sample is a urine or serum sample.

In some embodiments, the method further comprises comparing detection of the at least one Aspergillus species-associated molecule, such as an Af-associated molecule, in the sample obtained from the subject exhibiting one or more signs or symptoms associated with an Aspergillus species, such as Af to a control, wherein increased detection of the at least one Aspergillus species-associated molecule, such as the at least one Af-associated molecule, relative to a control indicates that the subject has Aspergillus species-associated condition, such as an Af-associated condition. In some examples, the method is used for diagnosing a subject with IA. In one example, the method is a method for monitoring the efficacy of therapy. In some examples, detecting at least one Af-associated molecule comprises using an enzyme-linked immunosorbent assay (ELISA). In some examples, the method is used for detecting any condition or disease associated with an Aspergillus species, such as Af.

The foregoing and other features will become more apparent from the following detailed description of several embodiments, which proceeds with reference to the accompanying figures.

### BRIEF DESCRIPTION OF THE FIGURES

FIG. 1 is a schematic illustration of an exemplary In vivo Antigen Discovery (InMAD strategy for identification of targets for immunoassay in aspergillosis.

FIG. 2 provides digital images of two-dimensional immunoblots of Aspergillus fumigatus lysate probed with InMAD immune serum. Mice were immunized with serum or urine from infected guinea pigs. Serum from the immunized mice was used to probe 2D blots of whole cell lysates of Af. Fifteen spots (proteins) not found after immunization with control serum or urine from uninfected animals are circled. Each spot is a candidate diagnostic target. Each number corresponds to the protein number in Table 1.

FIG. 3 is a schematic illustration of an exemplary strategy for validation of protein for immunoassay in diagnosis of aspergillosis.

FIG. 4 is a digital image of a two dimensional immunoblot of urine from guinea pig model of IA. The blot was probed with a cocktail of rabbit antibodies raised against predicted immunogenic peptides of Af: catalase, Ecm33, Eglc and thioreduxin. These correspond, respectively, to Proteins 1, 2, 3 and 5 in Table 1. Each spot was excised and the proteins were identified by LC-MS/MS. The proteomic analysis confirmed that these spots in urine from Af-infected guinea pigs were, in fact, the expected Af proteins. Taken together, this study provides validation of the overall target discovery process and identifies diagnostic targets for Aspergillus-associated conditions.

### DETAILED DESCRIPTION OF SEVERAL EMBODIMENTS

### I. Terms

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All GenBank and Protein ID Nos., publications, patent applications, patents, and other references mentioned herein are incorporated by reference in their entirety. In case of conflict, the present specification, including explanations of terms, will control. In addition, the materials, methods, and examples are illustrative only and not intended to be limiting.

In order to facilitate review of the various embodiments of this disclosure, the following explanations of specific terms are provided:

Adenoside deaminase: An enzyme involved in purine metabolism. A primary function of adenoside deaminase (also known as adenosine aminhydrolase or ADA) is the development and maintenance of the immune system. In some examples, adenoside deaminase is detected in subjects with Af. The term adenoside deaminase includes any adenoside deaminase gene, cDNA, mRNA, or protein from any organism. In one example, adenoside deaminase is used to detect and diagnosis an Aspergillus species-associated condition or disease, such as an Af-associated condition or disease.

Exemplary nucleic acid and protein sequences for adenoside deaminase are publicly available (see, GenBank Accession No. XM_{—}749022.2 (A. fumigatus) for an exemplary nucleic acid sequence, or NCBI Reference No. XP_{—}754115/GI:146323525 (A. fumigatus) or GenBank Accession No. EDP52248.1/GI:19912, for exemplary protein sequences (each of which hereby incorporated by reference as available on May 22, 2012).

In one example, adenoside deaminase includes a full-length wild-type (or native) sequence, as well as adenoside deaminase allelic variants, fragments, homologs or fusion sequences that retain the ability to be detected in a subject with Af. In certain examples, adenoside deaminase has at least 80% sequence identity, for example at least 85%, 90%, 95%, or 98% sequence identity to a known adenoside deaminase and retains adenoside deaminase activity (e.g., the capability to be detected in a subject with an Aspergillus species-associated condition or disease, such as an Af-associated condition or disease).

Adenosyl homocysteinase: An enzyme which catalyzes the hydrolysis of S-adenosyl-L-homocysteine (AdoHyc) to form adenosine (Ado) and homocysteine (Hcy). In some examples, adenosyl homocysteinase is detected in subjects with Af. The term adenosyl homocysteinase includes any adenosyl homocysteinase gene, cDNA, mRNA, or protein from any organism. In one example, adenosyl homocysteinase is used to detect and diagnosis an Aspergillus species-associated condition or disease, such as an Af-associated condition or disease.

Exemplary nucleic acid and protein sequences for adenosyl homocysteinase are publicly available (see, GenBank Accession No. XM_{—}747286.1 (A. fumigatus) for an exemplary nucleic acid sequence, or NCBI Reference No. XP_{—}75239/GI:7099521 (A. fumigatus) or Table 1 and GenBank Accession No. EDP56246.1/GI:159131133, for exemplary protein sequences (each of which hereby incorporated by reference as available on May 22, 2012)).

In one example, adenosyl homocysteinase includes a full-length wild-type (or native) sequence, as well as adenosyl homocysteinase allelic variants, fragments, homologs or fusion sequences that retain the ability to be detected in a subject with Af. In certain examples, adenosyl homocysteinase has at least 80% sequence identity, for example at least 85%, 90%, 95%, or 98% sequence identity to a known adenosyl homocysteinase and retains adenosyl homocysteinase activity (e.g., the capability to be detected in a subject with an Aspergillus species-associated condition or disease, such as an Af-associated condition or disease).

Adjuvant: A vehicle used to enhance antigenicity. Adjuvants include a suspension of minerals (alum, aluminum hydroxide, or phosphate) on which antigen is adsorbed; or water-in-oil emulsion in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). Immunstimulatory oligonucleotides (such as those including a CpG motif) can also be used as adjuvants (for example see U.S. Pat. No. 6,194,388; U.S. Pat. No. 6,207,646; U.S. Pat. No. 6,214,806; U.S. Pat. No. 6,218,371; U.S. Pat. No. 6,239,116; U.S. Pat. No. 6,339,068; U.S. Pat. No. 6,406,705; and U.S. Pat. No. 6,429,199). Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L and 41 BBL.

Animal: Living multi-cellular vertebrate organisms, a category that includes, for example, mammals and birds. The term mammal includes both human and non-human mammals. Similarly, the term “subject” includes both human and veterinary subjects, for example mice.

Antibody: A polypeptide ligand comprising at least a light chain or heavy chain immunoglobulin variable region which specifically binds an epitope of a protein listed in the tables below, or a fragment of any of these proteins. Antibodies can include a heavy chain and a light chain, each of which has a variable region, termed the variable heavy (VH) region and the variable light (VL) region. Together, the VH region and the VL region are responsible for binding the antigen recognized by the antibody. This includes intact immunoglobulins and the variants and portions of them well known in the art, such as Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes recombinant forms such as chimeric or humanized antibodies that may be derived from a murine antibody, heteroconjugate antibodies (such as, bispecific antibodies). See also, Pierce Catalog and Handbook, 1994-1995 (Pierce Chemical Co., Rockford, Ill.); Kuby, Immunology, 3rd Ed., W.H. Freeman & Co., New York, 1997.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. These fused cells and their progeny are termed “hybridomas.” Monoclonal antibodies include humanized monoclonal antibodies.

A variety of immunoassay formats are appropriate for selecting antibodies specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

β(1,3)-glucanosyltransferase: An enzyme which catalyzes the addition of β(1-3)glucans to create a new β(1-3) linkage. Glucans are a major component of fungal cell walls. In Af, the cell wall is composed of many polysaccharides, one of which is β(1-3)glucan. In some examples, β(1,3)-glucanosyltransferase is detected in subjects with Af. The term β(1,3)-glucanosyltransferase includes any β(1,3)-glucanosyltransferase gene, cDNA, mRNA, or protein from any organism. In one example, β(1,3)-glucanosyltransferase, such as Af β(1,3)-glucanosyltransferase, is used to detect and diagnose Af.

Exemplary nucleic acid and protein sequences for β(1,3)-glucanosyltransferase are publicly available (see, GenBank Accession Nos. 3506753 (A. fumigatus), 330879, (A. fumigatus) for exemplary nucleic acid sequences, or Table 1 and XP_{—}749664 (A. fumigatus), POC956, (A. fumigatus), for exemplary protein sequences (each of which hereby incorporated by reference as available on May 23, 2011)).

In one example, β(1,3)-glucanosyltransferase includes a full-length wild-type (or native) sequence, as well as β(1,3)-glucanosyltransferase allelic variants, fragments, homologs or fusion sequences that retain the ability to be detected in a subject with Af. In certain examples, β(1,3)-glucanosyltransferase has at least 80% sequence identity, for example at least 85%, 90%, 95%, or 98% sequence identity to a known β(1,3)-glucanosyltransferase and retains β(1,3)-glucanosyltransferase activity (e.g., the capability to be detected in a subject with an Aspergillus species-associated condition or disease, such as an Af-associated condition or disease).

Binding: A specific interaction between two or more molecules, such as the binding of an antibody and an antigen (for example an antibody to an antigen). In one embodiment, specific binding is identified by a dissociation constant (K_{d}). In one embodiment, binding affinity is calculated by a modification of the Scatchard method described by Frankel et al., Mol. Immunol., 16:101-106, 1979. In another embodiment, binding affinity is measured by an antigen/antibody dissociation rate. In yet another embodiment, a high binding affinity is measured by an ELISA. In another embodiment, binding association and dissociation rates are measured by surface plasmon resonance. In several examples, a high binding affinity is at least about 1×10⁻⁸ M. In other embodiments, a high binding affinity is at least about 1.5×10⁻⁸, at least about 2.0×10⁻⁸, at least about 2.5×10⁻⁸, at least about 3.0×10⁻⁸, at least about 3.5×10⁻⁸, at least about 4.0×10⁻⁸, at least about 4.5×10⁻⁸, or at least about 5.0×10⁻⁸ M. In one example, the disclosed antibodies have a binding affinity for the Aspergillus species-associated antigen, such as an Af-associated antigen of at least 10 nM.

Catalase: An enzyme found in nearly all living organisms that are exposed to oxygen, where it functions to catalyze the decomposition of hydrogen peroxide to water and oxygen Catalase has one of the highest turnover numbers of all enzymes; one catalase enzyme can convert 40 million molecules of hydrogen peroxide to water and oxygen each second. Catalase is a tetramer of four polypeptide chains, each over 500 amino acids long. It contains four porphyrin heme (iron) groups that allow the enzyme to react with the hydrogen peroxide. The optimum pH for human catalase is approximately 7, and has a fairly broad maximum (the rate of reaction does not change appreciably at pHs between 6.8 and 7.5). The pH optimum for other catalases varies between 4 and 11 depending on the species. The optimum temperature also varies by species.

In some examples, Af catalase is detected in subjects with an Af-associated condition, such as IA. The term catalase includes any catalase gene, cDNA, mRNA, or protein from any organism. In one example, catalase is used to detect and diagnose an Aspergillus species-associated condition or disease, such as an Af-associated condition or disease.

Exemplary nucleic acid and protein sequences for catalase are publicly available (see, GenBank Accession Nos. NM_{—}001752.3 (human), NM_{—}009804 (mouse), or U87850 (A. fumigatus), XM_{—}744063 (A. fumigatus), XM_{—}743457 (A. fumigatus) or XM_{—}742595 (A. fumigatus) for exemplary nucleic acid sequences and Table 1, NP_{—}001743 (human), NP_{—}033904 (mouse), AAB48485 (A. fumigatus), AAB71223 (A. fumigatus), CAA69069 (A. fumigatus), XP_{—}749156 (A. fumigatus), XP_{—}756140 (A. fumigatus), EAL94102 (A. fumigatus), XP_{—}747688 (A. fumigatus), or EAL85650 (A. fumigatus), for exemplary protein sequences (each of which hereby incorporated by reference as available on May 23, 2011)).

In one example, catalase includes a full-length wild-type (or native) sequence, as well as catalase allelic variants, fragments, homologs or fusion sequences that retain the ability to be detected in a subject with an Aspergillus species-associated condition or disease, such as an Af-associated condition or disease. In certain examples, catalase has at least 80% sequence identity, for example at least 85%, 90%, 95%, or 98% sequence identity to a known catalase and retains catalase activity (e.g., the capability to be detected in a subject with an Aspergillus species-associated condition or disease, such as an Af-associated condition or disease).

Chr-like protein: In some examples, chr-like protein is detected in subjects with Af. The term chr-like protein includes any chr-like protein gene, cDNA, mRNA, or protein from any organism. In one example, chr-like protein is used to detect and diagnosis an Aspergillus species-associated condition or disease, such as an Af-associated condition or disease.

Exemplary nucleic acid and protein sequences for chr-like protein are publicly available (see, GenBank Accession No. AY169706 (A. fumigatus) for an exemplary nucleic acid sequences or Table 1 and GenBank Accession No. AAN87849.1/GI:27372089, for exemplary protein sequences (each of which hereby incorporated by reference as available on May 22, 2012)).

In one example, chr-like protein includes a full-length wild-type (or native) sequence, as well as chr-like protein allelic variants, fragments, homologs or fusion sequences that retain the ability to be detected in a subject with Af. In certain examples, chr-like protein has at least 80% sequence identity, for example at least 85%, 90%, 95%, or 98% sequence identity to a known chr-like protein and retains chr-like protein activity (e.g., the capability to be detected in a subject with an Aspergillus species-associated condition or disease, such as an Af-associated condition or disease).

Conserved hypothetical protein 159122886: In some examples, conserved hypothetical protein 159122886 is detected in subjects with Af. The term conserved hypothetical protein 159122886 includes any conserved hypothetical protein 159122886 gene, cDNA, mRNA, or protein from any organism. In one example, conserved hypothetical protein 159122886 is used to detect and diagnose an Aspergillus species-associated condition or disease, such as an Af-associated condition or disease.

Exemplary nucleic acid and protein sequences for conserved hypothetical protein 159122886 are publicly available (see, GenBank Accession No. DS499601.1 (A. fumigatus) for an exemplary nucleic acid sequences or Table 1 and/or GenBank Accession No. EDP48006/GI:159122886, for exemplary protein sequences (each of which hereby incorporated by reference as available on May 22, 2012)).

In one example, conserved hypothetical protein 159122886 includes a full-length wild-type (or native) sequence, as well as conserved hypothetical protein 159122886 allelic variants, fragments, homologs or fusion sequences that retain the ability to be detected in a subject with Af. In certain examples, conserved hypothetical protein 159122886 has at least 80% sequence identity, for example at least 85%, 90%, 95%, or 98% sequence identity to a known conserved hypothetical protein 159122886 and retains conserved hypothetical protein 159122886 activity (e.g., the capability to be detected in a subject with an Aspergillus species-associated condition or disease, such as an Af-associated condition or disease).

Conserved hypothetical protein 70995516: In some examples, conserved hypothetical protein 70995516 is detected in subjects with Af. The term conserved hypothetical protein 70995516 includes any conserved hypothetical protein 70995516 gene, cDNA, mRNA, or protein from any organism. In one example, conserved hypothetical protein 70995516 is used to detect and diagnose an Aspergillus species-associated condition or disease, such as an Af-associated condition or disease.

Exemplary nucleic acid and protein sequences for conserved hypothetical protein 70995516 are publicly available (see, NCBI Ref. No. XM_{—}747420.1 (A. fumigatus) for an exemplary nucleic acid sequences or NCBI Reference No. XP_{—}752513.1/GI: 70995516 or Table 1 and/or GenBank Ref. No. EDP56381.1/GI:159131268, for exemplary protein sequences (each of which hereby incorporated by reference as available on May 22, 2012)).

In one example, conserved hypothetical protein 70995516 includes a full-length wild-type (or native) sequence, as well as conserved hypothetical protein 70995516 allelic variants, fragments, homologs or fusion sequences that retain the ability to be detected in a subject with Af. In certain examples, conserved hypothetical protein 70995516 has at least 80% sequence identity, for example at least 85%, 90%, 95%, or 98% sequence identity to a known conserved hypothetical protein 70995516 and retains conserved hypothetical protein 70995516 activity (e.g., the capability to be detected in a subject with an Aspergillus species-associated condition or disease, such as an Af-associated condition or disease).

Contacting: “Contacting” includes in solution and solid phase, for example contacting a salivary protein with a test agent. The test agent may also be a combinatorial library for screening a plurality of compounds. In another example, contacting includes contacting a sample with an antibody, for example contacting a sample that contains a protein of interest such as a protein associated with an Aspergillus species-associated condition or disease, such as an Af-associated condition or disease, such as one or more proteins provided in Table 1.

GPI-anchored cell wall beta 1,3-endoglucanase Eglc: A protein secreted from A. fumigatus. In some examples, Af GPI-anchored cell wall beta 1,3-endoglucanase Eglc is detected in subjects with an Af-associated condition. The term GPI-anchored cell wall beta 1,3-endoglucanase Eglc includes any GPI-anchored cell wall beta 1,3-endoglucanase Eglc gene, cDNA, mRNA, or protein from any organism. In one example, GPI-anchored cell wall beta 1,3-endoglucanase Eglc, such as Af GPI-anchored cell wall beta 1,3-endoglucanase Eglc is used to detect and diagnose an Aspergillus species-associated condition or disease, such as an Af-associated condition or disease.

Exemplary nucleic acid and protein sequences for GPI-anchored cell wall beta 1,3-endoglucanase Eglc are publicly available (see, GenBank Accession Nos. AFUA_{—}3 G00270 (A. fumigatus), AF1211332 (A. fumigatus) for exemplary nucleic acid sequences, or Table 1, XP_{—}748349 (A. fumigatus), and/or AAF13033.2 (A. fumigatus), for exemplary protein sequences (each of which hereby incorporated by reference as available on May 23, 2011)).

In one example, GPI-anchored cell wall beta 1,3-endoglucanase Eglc includes a full-length wild-type (or native) sequence, as well as GPI-anchored cell wall beta 1,3-endoglucanase Eglc allelic variants, fragments, homologs or fusion sequences that retain the ability to be detected in a subject with an Aspergillus species-associated condition or disease, such as an Af-associated condition or disease. In certain examples, GPI-anchored cell wall beta 1,3-endoglucanase Eglc has at least 80% sequence identity, for example at least 85%, 90%, 95%, or 98% sequence identity to a known GPI-anchored cell wall beta 1,3-endoglucanase Eglc and retains GPI-anchored cell wall beta 1,3-endoglucanase Eglc activity (e.g., the capability to be detected in a subject with an Aspergillus species-associated condition or disease, such as an Af-associated condition or disease).

GPI-anchored cell wall organization protein Ecm33: A protein involved in Af-cell wall morphogenesis; Af mutants with deletions in Ecm33 are associated with rapid conidial germination. In addition, the mutants show increased cell-cell adhesion and increased pathogenicity in a mouse model of aspergillosis. In some examples, Af GPI-anchored cell wall organization protein Ecm33 is detected in subjects with an Af-associated condition. The term GPI-anchored cell wall organization protein Ecm33 includes any GPI-anchored cell wall organization protein Ecm33 gene, cDNA, mRNA, or protein from any organism. In one example, GPI-anchored cell wall organization protein Ecm33, such as Af GPI-anchored cell wall organization protein Ecm33 is used to detect and diagnose an Aspergillus species-associated condition or disease, such as an Af-associated condition or disease.

Exemplary nucleic acid and protein sequences for GPI-anchored cell wall organization protein Ecm33 are publicly available (see, GenBank Accession Nos. AFUA_{—}4 G06820 (A. fumigatus), AFUB_{—}063890A (A. fumigatus), AFUA_{—}4 G06820 (A. fumigatus) for exemplary nucleic acid sequences, or Table 1, XP_{—}752144 (A. fumigatus), EDP50058.1 (A. fumigatus), and/or EAL90106.1 (A. fumigatus), for exemplary protein sequences (each of which hereby incorporated by reference as available on May 23, 2011)).

In one example, GPI-anchored cell wall organization protein Ecm33 includes a full-length wild-type (or native) sequence, as well as GPI-anchored cell wall organization protein Ecm33 allelic variants, fragments, homologs or fusion sequences that retain the ability to be detected in a subject with an Aspergillus species-associated condition. In certain examples, GPI-anchored cell wall organization protein Ecm33 has at least 80% sequence identity, for example at least 85%, 90%, 95%, or 98% sequence identity to a known GPI-anchored cell wall organization protein Ecm33 and retains GPI-anchored cell wall organization protein Ecm33 (e.g., the capability to be detected in a subject with an Aspergillus species-associated condition or disease, such as an Af-associated condition or disease).

Fungus: Living, single-celled and multicellular organisms belonging to the kingdom Fungi. Most species are characterized by a lack of chlorophyll and presence of chitinous cell walls, and some fungi may be multinucleated. In one example, a fungus is an Aspergillus species. Representative, non-limiting examples of Aspergillus species include A. candidus, A. chevalieri, A. clavatus, A. flavipes, A. flavus, A. fumigatus, A. granulosus, A. nidulans, A. niger, A. parasiticus, A. restrictus, A. sydowii, A. tamari, A. ustus, A. versicolor, and A. wentii.

In one example, the fungus is Aspergillus fumigatus (Af). Aspergillus fumigatus is one of the most common Aspergillus species to cause disease in individuals with an immunodeficiency. A. fumigatus, a saprotroph widespread in nature, is typically found in soil and decaying organic matter, such as compost heaps, where it plays an essential role in carbon and nitrogen recycling. Colonies of the fungus produce from conidiophores thousands of minute grey-green conidia (2-3 μm) that readily become airborne. The fungus is capable of growth at 37° C./99° F., and can grow at temperatures up to 50° C./122° F., with conidia surviving at 70° C./158° F.—conditions it regularly encounters in self-heating compost heaps. Its spores are ubiquitous in the atmosphere. In immunocompromised individuals, such as organ or bone marrow transplant recipients and people with leukemia, the fungus is more likely to become pathogenic, over-running the host's weakened defenses and causing a range of diseases generally termed aspergillosis.

An “Aspergillus species-associated molecule” is a molecule associated with one or more signs or symptoms of an Aspergillus species-associated condition or disease. In some examples, an Aspergillus species-associated molecule is one or more of the antigens provided in Table 1. In some examples, an “Aspergillus species-associated condition or disease” is one which is associated with the particular Aspergillus species. In some examples, an“Af-associated molecule” is a molecule associated with one or more signs or symptoms of an Af-associated condition or disease. In some examples, an Af-associated molecule is one or more of the antigens provided in Table 1. In some examples, an “Af-associated condition or disease” is one which is associated with Af, such as aspergillosis, including, but not limited to IA.

Glu/Leu/Phe/Val (ELFV) Dehydrogenase: A family of enzymes which includes glutamate, leucine, phenylalanine and valine dehydrogenases. These enzymes are structurally and functionally related. They contain a Gly-rich region containing a conserved Lys residue, which has been implicated in the catalytic activity, in each case a reversible oxidative deamination reaction.

In some examples, glu/leu/phe/val dehydrogenase is detected in subjects with Af. The term glu/leu/phe/val dehydrogenase includes any glu/leu/phe/val dehydrogenase gene, cDNA, mRNA, or protein from any organism. In one example, glu/leu/phe/val dehydrogenase is used to detect and diagnose an Aspergillus species-associated condition or disease, such as an Af-associated condition or disease.

Exemplary nucleic acid and protein sequences for glu/leu/phe/val dehydrogenase are publicly available (see, Table 1, NCBI Reference No. XP_{—}752164.1/GI: 70994774, for exemplary protein sequences which is hereby incorporated by reference as available on May 22, 2012)).

In one example, glu/leu/phe/val dehydrogenase includes a full-length wild-type (or native) sequence, as well as glu/leu/phe/val dehydrogenase allelic variants, fragments, homologs or fusion sequences that retain the ability to be detected in a subject with Af. In certain examples, glu/leu/phe/val dehydrogenase has at least 80% sequence identity, for example at least 85%, 90%, 95%, or 98% sequence identity to a known glu/leu/phe/val dehydrogenase and retains glu/leu/phe/val dehydrogenase activity (e.g., the capability to be detected in a subject with an Aspergillus species-associated condition or disease, such as an Af-associated condition or disease).

Immunoassay: A biochemical test that measures the presence or concentration of a substance in a sample, such as a biological sample, using the reaction of an antibody to its cognate antigen, for example the specific binding of an antibody to a protein. Both the presence of antigen and the amount of antigen present can be measured. For measuring proteins, for each the antigen and the presence and amount (abundance) of the protein can be determined or measured. Measuring the quantity of antigen can be achieved by a variety of methods. One of the most common is to label either the antigen or antibody with a detectable label.

An “enzyme linked immunosorbent assay (ELISA)” is type of immunoassay used to test for antigens (for example, proteins present in a sample, such as a biological sample). A “competitive radioimmunoassay (RIA)” is another type of immunoassay used to test for antigens. A “lateral flow immunochromatographic (LFI)” assay is another type of immunoassay used to test for antigens.

Immunogenic peptide: A peptide which comprises an allele-specific motif or other sequence such that the peptide will bind an MHC molecule and induce a cytotoxic T lymphocyte (“CTL”) response, or a B cell response (e.g., antibody production) against the antigen from which the immunogenic peptide is derived.

In one embodiment, immunogenic peptides are identified using sequence motifs or other methods, such as neural net or polynomial determinations, known in the art. Typically, algorithms are used to determine the “binding threshold” of peptides to select those with scores that give them a high probability of binding at a certain affinity and will be immunogenic. The algorithms are based either on the effects on MHC binding of a particular amino acid at a particular position, the effects on antibody binding of a particular amino acid at a particular position, or the effects on binding of a particular substitution in a motif-containing peptide. Within the context of an immunogenic peptide, a “conserved residue” is one which appears in a significantly higher frequency than would be expected by random distribution at a particular position in a peptide. In one embodiment, a conserved residue is one where the MHC structure may provide a contact point with the immunogenic peptide.

Immunogenic peptides can also be identified by measuring their binding to a specific MHC protein (e.g., HLA-A02.01) and by their ability to stimulate CD4 and/or CD8 when presented in the context of the MHC protein. The characteristics of immunogenic polypeptides, are disclosed, for example, in PCT Publication No. WO 00/12706, which is incorporated herein by reference.

In one example, an immunogenic “Af peptide” is a series of contiguous amino acid residues from an Af-associated protein, such as an antigen provided in Table 1, generally between 7 and 20 amino acids in length, such as about 8 to 15 residues in length. Generally, immunogenic Af polypeptides can be used to induce an immune response in a subject, such as a B cell response or a T cell response. In one example, an immunogenic Af polypeptide, when bound to a Major Histocompatibility Complex Class I molecule, activates cytotoxic T lymphocytes (CTLs) against cells expressing wild-type Af associated protein, such as one or more antigens listed in Table 1. Induction of CTLs using synthetic peptides and CTL cytotoxicity assays known in the art, see U.S. Pat. No. 5,662,907, which is incorporated herein by reference. In one example, an immunogenic peptide includes an allele-specific motif or other sequence such that the peptide will bind an MHC molecule and induce a cytotoxic T lymphocyte (“CTL”) response against the antigen from which the immunogenic peptide is derived.

Immunogenic composition: A composition comprising an immunogenic Aspergillus species polypeptide, such as an Af polypeptide or a nucleic acid encoding the immunogenic Af-associated polypeptide that induces a measurable CTL response against cells expressing Af polypeptide, or induces a measurable B cell response against a Af-associated polypeptide. For in vitro use, the immunogenic composition can consist of the isolated nucleic acid, vector including the nucleic acid/or immunogenic peptide. For in vivo use, the immunogenic composition will typically comprise the nucleic acid, vector including the nucleic acid, and or immunogenic polypeptide, in pharmaceutically acceptable carriers, and/or other agents. An immunogenic composition can optionally include an adjuvant, a costimulatory molecule, or a nucleic acid encoding a costimulatory molecule. An Aspergillus species polypeptide, or nucleic acid encoding the polypeptide, can be readily tested for its ability to induce a CTL by art-recognized assays.

Inhibiting or treating a condition or disease: A phrase used to refer to inhibiting the full development of a disease, such as an Aspergillus species-associated condition, such as Af-associated condition, including IA. In several examples, inhibiting a disease refers to lessening symptoms of the Aspergillus species-associated condition, such as of the Af-associated condition or disease. “Treatment” refers to a therapeutic intervention that ameliorates a sign or symptom of a disease or pathological condition related to the disease, such as the Aspergillus species-associated disease.

Invasive aspergillosis (IA): An opportunistic fungal infection caused mainly by Aspergillus fumigatus (Af). Invasive aspergillosis normally only occurs in severely immune-compromised patients and has a high mortality rate (25-90%). Invasive disease is most commonly seen in the lungs, which is called pulmonary aspergillosis, but although less common, dissemination of aspergillus to other tissues, including the central nervous system, sinuses, bone, heart, kidney, eye, blood and skin, has been reported.

Risk factors for invasive aspergillosis include patients on steroids, chemotherapy treatment resulting in severe neutropenia, stem cell and solid organ transplantation, later stages of AIDS, and a genetic disease called chronic granulomatous disease.

Diagnosis can be made by detection of aspergillus species by biopsy, culture and microscopy of tissue samples. Chest CT scans and detection of aspergillus antigens in body fluids, by a variety of methods, all assist with diagnosis but have a relatively low sensitivity and specificity for diagnosis of invasive aspergillosis. The assay and methods disclosed herein have a high degree of sensitivity and specificity for diagnosis of invasive aspergillosis.

Label: A detectable compound or composition that is conjugated directly or indirectly to another molecule, such as an antibody or a protein, to facilitate detection of that molecule. Specific, non-limiting examples of labels include fluorescent tags, enzymatic linkages (such as horseradish peroxidase), radioactive isotopes (for example ¹⁴C, ³²P, ¹²⁵I, ³H isotopes and the like) and particles such as colloidal gold. In some examples a protein, such as a protein associated with an Af-associated condition, is labeled with a radioactive isotope, such as ¹⁴C, ³²P, ¹²⁵I, ³H isotope. In some examples an antibody that specifically binds the protein is labeled. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998), Harlow & Lane (Antibodies, A Laboratory Manual, Cold Spring Harbor Publications, New York, 1988).

L-amino acid oxidase Lao (LAO): An enzyme which catalyzes the oxidative deamination of a number of L-amino acids. This enzyme has been shown to play role in the purification and determination of certain amino acids and the preparation of a-keto acid

In some examples, L-amino acid oxidase Lao is detected in subjects with Af. The term L-amino acid oxidase Lao includes any L-amino acid oxidase Lao gene, cDNA, mRNA, or protein from any organism. In one example, L-amino acid oxidase Lao is used to detect and diagnose an Aspergillus species-associated condition or disease, such as an Af-associated condition or disease.

Exemplary nucleic acid and protein sequences for L-amino acid oxidase Lao are publicly available (see, GenBank No. EAL86792.1 or Table 1 and/or NCBI Ref No. XP_{—}748830.1/GI:70986680, for exemplary protein sequences (each of which hereby incorporated by reference as available on May 22, 2012)).

In one example, L-amino acid oxidase Lao includes a full-length wild-type (or native) sequence, as well as L-amino acid oxidase Lao allelic variants, fragments, homologs or fusion sequences that retain the ability to be detected in a subject with Af. In certain examples, L-amino acid oxidase Lao has at least 80% sequence identity, for example at least 85%, 90%, 95%, or 98% sequence identity to a known L-amino acid oxidase Lao and retains L-amino acid oxidase Lao activity (e.g., the capability to be detected in a subject with an Aspergillus species-associated condition or disease, such as an Af-associated condition or disease).

Major allergen Asp f 2: Asp f 2 is a highly immunogenic antigen that reacts with IgE antibodies in patients with allergic bronchopulmonary aspergillosis. The protein also binds to laminin and therefore, may be involved in Af adherence to the extracellular matrix. In some examples, major allergen Asp f 2 is detected in subjects with Af. The term major allergen Asp f 2 includes any thioreduxin reductase GLiT gene, cDNA, mRNA, or protein from any organism. In one example, major allergen Asp f 2 is used to detect and diagnosis an Aspergillus species-associated condition or disease, such as an Af-associated condition or disease.

Exemplary nucleic acid and protein sequences for major allergen Asp f 2 are publicly available (see, GenBank Accession No. AFUA_{—}4 G09580 (A. fumigatus), for an exemplary nucleic acid sequence, or Table 1 and/or P79017 (A. fumigatus), for exemplary protein sequences (each of which hereby incorporated by reference as available on May 23, 2011)).

In one example, major allergen Asp f 2 includes a full-length wild-type (or native) sequence, as well as major allergen Asp f 2 allelic variants, fragments, homologs or fusion sequences that retain the ability to be detected in a subject with Af. In certain examples, major allergen Asp f 2 has at least 80% sequence identity, for example at least 85%, 90%, 95%, or 98% sequence identity to a known major allergen Asp f 2 and retains major allergen Asp f 2 activity (e.g., the capability to be detected in a subject with an Aspergillus species-associated condition or disease, such as an Af-associated condition or disease).

Peptide Modifications: Af-associated peptides include synthetic embodiments of peptides described herein. In addition, analogs (non-peptide organic molecules), derivatives (chemically functionalized peptide molecules obtained starting with the disclosed peptide sequences) and variants (homologs) of these proteins can be utilized in the methods described herein. Each polypeptide of this disclosure is comprised of a sequence of amino acids, which may be either L- and/or D-amino acids, naturally occurring and otherwise.

Peptides can be modified by a variety of chemical techniques to produce derivatives having essentially the same activity as the unmodified peptides, and optionally having other desirable properties. For example, carboxylic acid groups of the protein, whether carboxyl-terminal or side chain, can be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring. Amino groups of the peptide, whether amino-terminal or side chain, can be in the form of a pharmaceutically-acceptable acid addition salt, such as the HCl, HBr, acetic, benzoic, toluene sulfonic, maleic, tartaric and other organic salts, or can be modified to C₁-C₁₆ alkyl or dialkyl amino or further converted to an amide.

Hydroxyl groups of the peptide side chains may be converted to C₁-C₁₆ alkoxy or to a C₁-C₁₆ ester using well-recognized techniques. Phenyl and phenolic rings of the peptide side chains may be substituted with one or more halogen atoms, such as fluorine, chlorine, bromine or iodine, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be protected with any one of a number of well-recognized protecting groups, such as acetamide groups. Those skilled in the art will also recognize methods for introducing cyclic structures into the peptides of this disclosure to select and provide conformational constraints to the structure that result in enhanced stability.

Peptidomimetic and organomimetic embodiments are envisioned, whereby the three-dimensional arrangement of the chemical constituents of such peptido- and organomimetics mimic the three-dimensional arrangement of the peptide backbone and component amino acid side chains, resulting in such peptido- and organomimetics of an immunogenic Af polypeptide having measurable or enhanced ability to generate an immune response. For computer modeling applications, a pharmacophore is an idealized three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs,” in Klegerman & Groves, eds., 1993, Pharmaceutical Biotechnology, Interpharm Press: Buffalo Grove, Ill., pp. 165-174 and Principles of Pharmacology, Munson (ed.) 1995, Ch. 102, for descriptions of techniques used in CADD. Also included are mimetics prepared using such techniques.

Peptidyl-prolyl cis-trans isomerase: An enzyme (also known as prolyl isomerase) found in both prokaryotes and eukaryotes that interconverts the cis and trans isomers of peptide bonds with the amino acid proline. In some examples, peptidyl-prolyl cis-trans isomerase is detected in subjects with Af. The term peptidyl-prolyl cis-trans isomerase includes any peptidyl-prolyl cis-trans isomerase gene, cDNA, mRNA, or protein from any organism. In one example, peptidyl-prolyl cis-trans isomerase is used to detect and diagnosis an Aspergillus species-associated condition or disease, such as an Af-associated condition or disease.

Exemplary nucleic acid and protein sequences for peptidyl-prolyl cis-trans isomerase are publicly available (see, NCBI Ref. No. XM_{—}744411.1 (A. fumigatus) for an exemplary nucleic acid sequences or NCBI Reference No. XP_{—}749504.1/GI: 70989309 or Table 1 and/or GenBank Ref. No. EDP54029/GI:159128915, for exemplary protein sequences (each of which hereby incorporated by reference as available on May 22, 2012)).

In one example, peptidyl-prolyl cis-trans isomerase includes a full-length wild-type (or native) sequence, as well as peptidyl-prolyl cis-trans isomerase allelic variants, fragments, homologs or fusion sequences that retain the ability to be detected in a subject with Af. In certain examples, peptidyl-prolyl cis-trans isomerase has at least 80% sequence identity, for example at least 85%, 90%, 95%, or 98% sequence identity to a known peptidyl-prolyl cis-trans isomerase and retains peptidyl-prolyl cis-trans isomerase activity (e.g., the capability to be detected in a subject with an Aspergillus species-associated condition or disease, such as an Af-associated condition or disease).

Pharmaceutically acceptable carriers: The pharmaceutically acceptable carriers of use are conventional. Remington's Pharmaceutical Sciences, by E. W. Martin, Mack Publishing Co., Easton, Pa., 19th Edition (1995), describes compositions and formulations suitable for pharmaceutical delivery of the Af-associated peptides herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pigment Biosynthesis Protein: A protein involved in pigment biosynthesis. In some examples, pigment biosynthesis protein is detected in subjects with Af. The term pigment biosynthesis protein includes any pigment biosynthesis protein gene, cDNA, mRNA, or protein from any organism. In one example, pigment biosynthesis protein is used to detect and diagnosis an Aspergillus species-associated condition or disease, such as an Af-associated condition or disease.

Exemplary nucleic acid and protein sequences for pigment biosynthesis protein are publicly available (see, GenBank Ref. No. FJ406493.1/GI:211909650 (A. fumigatus) for an exemplary nucleic acid sequences or Table 1 and/or GenBank Ref. No. ACJ13064.1/GI:211909651, for exemplary protein sequences (each of which hereby incorporated by reference as available on May 22, 2012)).

In one example, pigment biosynthesis protein includes a full-length wild-type (or native) sequence, as well as pigment biosynthesis protein allelic variants, fragments, homologs or fusion sequences that retain the ability to be detected in a subject with Af. In certain examples, pigment biosynthesis protein has at least 80% sequence identity, for example at least 85%, 90%, 95%, or 98% sequence identity to a known pigment biosynthesis protein and retains pigment biosynthesis protein activity (e.g., the capability to be detected in a subject with an Aspergillus species-associated condition or disease, such as an Af-associated condition or disease).

Polynucleotide: The term polynucleotide or nucleic acid sequence refers to a polymeric form of nucleotide at least 10 bases in length. A recombinant polynucleotide includes a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides can be ribonucleotides, deoxyribonucleotides, or modified forms of either nucleotide. The term includes single- and double-stranded forms of DNA.

Polypeptide: Any chain of amino acids, regardless of length or post-translational modification (e.g., glycosylation or phosphorylation). In one embodiment, the polypeptide is an Af-associated polypeptide. A polypeptide can be between 3 and 30 amino acids in length. In one embodiment, a polypeptide is from about 7 to about 25 amino acids in length. In yet another embodiment, a polypeptide is from about 8 to about 15 amino acids in length. In yet another embodiment, a peptide is about 14 amino acids in length. With regard to polypeptides, the word “about” indicates integer amounts. Thus, in one example, a polypeptide “about” 9 amino acids in length is from 8 to 10 amino acids in length.

Therapeutic agent: A substance that demonstrates some therapeutic effect by restoring or maintaining health, such as by alleviating the symptoms associated with a disease or physiological disorder, or delaying (including preventing) progression or onset of a disease. In some instances, the therapeutic agent is a chemical or pharmaceutical agent, or a prodrug. A therapeutic agent may be an agent that prevents or inhibits one or more signs or symptoms or laboratory findings associated with an Aspergillus species-associated condition, such as an Af-associated condition, including IA.

A “therapeutically effective amount” or “therapeutically effective dose” is that amount or dose sufficient to inhibit or prevent onset or advancement, to treat outward symptoms, or to cause regression, of a disease. The therapeutically effective amount or dose also can be considered as that amount or dose capable of relieving symptoms caused by the disease. Thus, a therapeutically effective amount or dose of an anti-Aspergillus species agent, such as an anti-Af agent, is that amount or dose sufficient to achieve a stated therapeutic effect.

Thioreduxin reductase GliT: GliT has recently been shown to protect Af from gliotoxin. The protein has the ability to break a disulphide bond within gliotoxin, which may lead to protection by inactivation. In some examples, thioreduxin reductase GLiT is detected in subjects with Af. The term conserved thioreduxin reductase GLiT includes any thioreduxin reductase GLiT gene, cDNA, mRNA, or protein from any organism. In one example, thioreduxin reductase GLiT is used to detect and diagnosis an Aspergillus species-associated condition or disease, such as an Af-associated condition or disease.

Exemplary nucleic acid and protein sequences for thioreduxin reductase GliT are publicly available (see, GenBank Accession No. 3508168 (A. fumigatus), for an exemplary nucleic acid sequence, or Table 1 and/or XP_{—}750863 (A. fumigatus), for exemplary protein sequences (each of which hereby incorporated by reference as available on May 23, 2011)).

In one example, thioreduxin reductase GliT includes a full-length wild-type (or native) sequence, as well as thioreduxin reductase GliT allelic variants, fragments, homologs or fusion sequences that retain the ability to be detected in a subject with Af. In certain examples, thioreduxin reductase GliT has at least 80% sequence identity, for example at least 85%, 90%, 95%, or 98% sequence identity to a known thioreduxin reductase GliT and retains thioreduxin reductase GliT (e.g., the capability to be detected in a subject with an Aspergillus species-associated condition or disease, such as an Af-associated condition or disease).

### II. Overview of Several Embodiments

Disclosed herein are methods of diagnosing a subject with an Aspergillus species-associated condition, such as an Af-associated condition, or monitoring the efficacy of a therapy. In some embodiments, a method of diagnosing a subject with an Aspergillus species-associated condition, such as an Af-associated condition, or monitoring the efficacy of a therapy, comprises detecting at least one Aspergillus species-associated molecule, such as at least one Af-associated molecule, in a sample obtained from a subject exhibiting one or more signs or symptoms associated with an Aspergillus species-associated condition, such as an Af-associated condition, or a subject known to be at risk of acquiring an Aspergillus species-associated condition, such as a subject known to be at risk of acquiring an Af-associated condition, wherein the at least one Aspergillus species-associated molecule, such as at least one Af-associated molecule, is an Aspergillus species catalase (Protein ID No. 70986104), GPI-anchored cell wall β-1,3-endogluconase (Eglc) (Protein ID No. 70985687), GPI-anchored cell wall organization protein (Ecm33) (Protein ID No. 70994734), Chr-like protein (Protein ID No. 27372089), Thioreduxin reductase (Protein ID No. 70992029), Peptidyl-prolyl cis-trans isomerase (Protein ID No. 70989309), Major allergen (Protein ID No. 83300352), Conserved hypothetical protein (Protein ID No. 159122886), Conserved hypothetical protein (Protein ID No. 70995516), 1,3-β-glucanosyltransferase (Protein ID No. 70989629), Adenoside deaminase (Protein ID No. 146323525), L-amino acid oxidase Lao (Protein ID No. 70986680), Glu/Leu/Phe/Val Dehydrogenase (Protein ID No. 70994774), Adenosyl homocysteinase (Protein ID No. 70995231), Pigment biosynthesis protein (Protein ID No. 211909651).

In some embodiments, the method of diagnosing or monitoring the efficacy of a therapy further comprises comparing detection of the at least one Aspergillus species-associated molecule, such as the at least one Af-associated molecule, in the sample obtained from the subject exhibiting one or more signs or symptoms associated with an Aspergillus species-associated condition, such as an Af-associated condition, to a control, wherein increased detection of the at least one Aspergillus species-associated molecule, such as the at least one Af-associated molecule, relative to a control indicates that the subject has an Aspergillus species-associated condition, such as an Af-associated condition.

In some examples, detecting of the at least one Aspergillus species-associated molecule, such as the at least one Af-associated molecule, comprises usage of at least one antibody specific for the at least one Aspergillus species-associated molecule.

In some examples, detecting of the at least one Aspergillus species-associated molecule, such as the at least one Af-associated molecule, comprises usage of at least two antibodies specific for at least two different epitopes on a specific Aspergillus species-associated molecule.

In some examples, the disclosed methods are used for diagnosing a subject with invasive aspergillosis.

In some examples, the method is a method for monitoring the efficacy of therapy.

In some examples, detecting at least one Aspergillus species-associated molecule, such as at least one Af-associated molecule, comprises using an ELISA or a lateral flow device.

In some examples, the sample is a urine sample.

In some examples, the sample is a serum sample.

In some examples, the sample is a whole blood sample (e.g., from a finger prick).

In some examples, the sample is broncho-alveolar lavage fluid (BALF).

In some embodiments, the disclosed methods include detecting other Aspergillus species.

Also disclosed are kits for detecting an Aspergillus species-associated condition, such as an Af-associated condition. In some embodiments, a kit for detecting at an Aspergillus species-associated condition, such as an Af-associated condition, comprises an ELISA or lateral flow device capable of detecting at least one or more of the disclosed Aspergillus species-associated molecules, such as the disclosed Af-associated molecules presented in Table 1.

In some examples, the disclosed methods and kits are used for self monitoring in which a subject, such as an immunosuppressed subject, monitors the presence of one or Aspergillus species, such as Af, to monitor the onset of an infection. In additional examples, the disclosed methods and kits are used for self monitoring in which a subject, such as a subject that has previously been diagnosed and treated for an Aspergillus-species associated condition or disease, such as IA, practices the method or uses the kit to monitor for relapse.

### III. Methods for Detecting an Af-associated Condition and Monitoring the Efficacy of a Therapeutic Regimen

Methods are disclosed herein that are of use to determine if a subject has an Aspergillus species-associated condition, such as aspergillosis, including IA, or to monitor the efficacy of therapy. These methods utilize a biological fluid, such as, but not limited to urine or serum or BALF, for the detection of a molecule associated with an Aspergillus species-associated condition, such as IA, including, but not limited to, protein antigens including an Aspergillus Catalase (Protein ID No. 70986104), GPI-anchored cell wall β-1,3-endogluconase (Eglc) (Protein ID No. 70985687), GPI-anchored cell wall organization protein (Ecm33) (Protein ID No. 70994734), Chr-like protein (Protein ID No. 27372089), Thioreduxin reductase (Protein ID No. 70992029), Peptidyl-prolyl cis-trans isomerase (Protein ID No. 70989309), Major allergen (Protein ID No. 83300352), Conserved hypothetical protein (Protein ID No. 159122886), Conserved hypothetical protein (Protein ID No. 70995516), 1,3-β-glucanosyltransferase (Protein ID No. 70989629), Adenoside deaminase (Protein ID No. 146323525), L-amino acid oxidase Lao (Protein ID No. 70986680), Glu/Leu/Phe/Val Dehydrogenase (Protein ID No. 70994774), Adenosyl homocysteinase (Protein ID No. 70995231), Pigment biosynthesis protein (Protein ID No. 211909651) or any combination thereof. The Aspergillus species-associated molecules include any naturally occurring forms of the proteins, such as but not limited to glycosylated forms and degradation products of the Aspergillus species-associated molecules (e.g., measurement of degradation products of Aspergillus species-associated molecules in a urine sample). In some embodiments, the methods disclosed herein are used to identify a subject as having aspergillosis. In some embodiments, the methods are used to identify IA. These methods can be performed over time, to monitor the progression or regression of an Aspergillus species-associated condition or disease in a subject, or to assess for the development of an Aspergillus species-associated condition from a pre-Aspergillus species condition. In some examples, the disclosed methods are used for self monitoring in which a subject, such as an immunosuppressed subject, monitors the presence of one or Aspergillus species, such as Af, to monitor the onset of an infection. In additional examples, the disclosed methods and kits are used for self monitoring in which a subject, such as a subject that has previously been diagnosed and treated for an Aspergillus-species associated condition or disease, such as IA, practices the method or uses the kit to monitor for relapse.

Methods are disclosed herein that include testing a biological sample, such as a serum or urine sample, obtained from the subject. In one example, the biological sample is a biological fluid, such as urine. However, other biological fluids are also of use, such as blood (such as whole blood obtained from a finger prick), GCF, amniotic fluid, BALF, salvia or tears. The methods include detecting, or determining the abundance (amount) of one or more molecules associated with an Aspergillus species-associated condition, such as an Af-associated condition, including protein antigens listed in Table 1. In some examples, the methods include determining a proteomic profile. In one example, the method includes detecting at least one protein antigen selected from Aspergillus Catalase (Protein ID No. 70986104), GPI-anchored cell wall β-1,3-endogluconase (Eglc) (Protein ID No. 70985687), GPI-anchored cell wall organization protein (Ecm33) (Protein ID No. 70994734), Chr-like protein (27372089), Thioreduxin reductase (Protein ID No. 70992029), Peptidyl-prolyl cis-trans isomerase (Protein ID No. 70989309), Major allergen (Protein ID No. 83300352), Conserved hypothetical protein (Protein ID No. 159122886), Conserved hypothetical protein (70995516), 1,3-β-glucanosyltransferase (Protein ID No. 70989629), Adenoside deaminase (Protein ID No. 146323525), L-amino acid oxidase Lao (Protein ID No. 70986680), Glu/Leu/Phe/Val Dehydrogenase (70994774), Adenosyl homocysteinase (Protein ID No. 70995231), and Pigment biosynthesis protein (Protein ID No. 211909651).

In some examples, the methods include detecting at least one, such as at least two, at least two, at least three, at least four, at least five, at least six, at least seven, at least eight, at least nine, at least ten, at least eleven, at least twelve, at least thirteen, at least fourteen, such as one, two, three, four, five, six, seven, eight, nine, ten, eleven, twelve, thirteen, fourteen or fifteen molecules associated with an Aspergillus species-associated condition or disease, such as an Af-associated condition or disease. In one example, the method includes detecting at least one, such as at least two, at least two, at least three, at least four, at least five, at least six, at least seven, at least eight, at least nine, at least ten, at least eleven, at least twelve, at least thirteen, at least fourteen, such as one, two, three, four, five, six, seven, eight, nine, ten, eleven, twelve, thirteen, fourteen or all fifteen molecules listed in Table 1.

In some embodiments, the method includes detecting an increase, such as a statistically significant increase, such as an at least 1.5, at least 2, at least 3, at least 4, or at least 5 fold increase in the amount of one or more molecules associated with an Aspergillus species-associated condition or disease, including an Af-associated condition or disease, including at least a 1.5, at least a 2, at least a 3, at least a 4, or at least a 5, such as a 1.5, 2, 2.5, 3, 3.5, 4, 5 fold increase in one or more Aspergillus species-associated molecules, such as one or more Af protein antigens listed in Table 1 as compared to a reference value.

In one embodiment, the method includes comparing a proteomic profile of a test sample of urine from a subject of interest comprising at least one of protein associated with an Aspergillus species, such as an Af protein antigen listed in Table 1 or all of these molecules with a proteomic profile from a reference sample.

In one embodiment, the method determines if the subject has an Aspergillus species-associated condition or disease, such as an Af-associated condition. If the reference sample is a normal sample and the proteomic profile of the test sample is essentially the same as the proteomic profile of the normal sample, the subject is determined not to have an Aspergillus species-associated condition or disease. However, if the proteomic profile of the test sample has a unique expression signature relative to the proteomic profile of the normal sample the subject is determined to have an Aspergillus species-associated condition or disease.

In some embodiments, if the reference sample is a sample from a subject with an Aspergillus species-associated condition or disease, such as an Af-associated condition, and its proteomic profile shares at least one unique expression signature characteristic with the reference sample, then the subject is determined to have an Aspergillus species-associated condition or disease. If the proteomic profile of the test sample has a unique expression signature relative to the reference sample the subject is determined not to have an Aspergillus species-associated condition or disease, such as IA. Hence, the proteomic profile provides an additional diagnostic criterion for these disorders. In one embodiment, the method is a method to determine if a therapy is effective for the treatment of the subject by detecting the presence of at least one protein associated with Aspergillus species-associated condition or disease, such as an Af-associated condition or disease. The method can be performed multiple times over a specified time period, such as days, weeks, months or years. In several examples, the therapy includes treatment with a therapeutic agent for an Aspergillus species-associated condition or disease, such as a therapeutic agent for an Af-associated condition. If the reference sample is a normal sample, and the proteomic profile of the test sample is essentially the same as the proteomic profile of the normal sample the subject is determined to have an effective therapy, while if the proteomic profile of the test sample has a unique expression signature relative to the proteomic profile of the normal sample to have an ineffective therapy. If the reference sample is a sample from a subject with an Aspergillus species-associated condition or disease, including an Af-associated condition, and proteomic profile shares at least one unique expression signature characteristic with the reference sample then the subject is determined to have an ineffective therapy, while if the proteomic profile of the test sample has a unique expression signature relative to the reference sample the subject is determined to have an effective therapy. Changes in the profile can also represent the progression (or regression) of the disease process. Methods for monitoring the efficacy of therapeutic agents are described below.

### Monitoring

The diagnostic methods of the present disclosure are valuable tools for practicing physicians to make quick treatment decisions for an Aspergillus species-associated condition, such as aspergillosis, including IA. These treatment decisions can include the administration of an anti-Aspergillus species agent and decisions to monitor a subject for onset and/or advancement of an Aspergillus species-associated condition. The method disclosed herein can also be used to monitor the effectiveness of a therapy. This monitoring can be performed by a clinical healthcare provider or a patient themselves. For example, a subject, such as an immunosuppressed subject, uses a disclosed method to monitor the presence of one or Aspergillus species, such as Af, to monitor the onset of an infection. In additional examples, the disclosed methods and kits are used for self monitoring in which a subject, such as a subject that has previously been diagnosed and treated for an Aspergillus-species associated condition or disease, such as IA, practices the method to monitor for relapse.

Following the measurement of the expression levels of one or more of the molecules identified herein, the assay results, findings, diagnoses, predictions and/or treatment recommendations are typically recorded and communicated to technicians, physicians and/or patients, for example. In certain embodiments, computers will be used to communicate such information to interested parties, such as, patients and/or the attending physicians. Based on the measurement, the therapy administered to a subject can be modified or started (in the case of monitoring for a relapse).

In one embodiment, a diagnosis, prediction and/or treatment recommendation based on the expression level in a test subject of one or more of the Aspergillus species-associated molecules disclosed herein is communicated to the subject as soon as possible after the assay is completed and the diagnosis and/or prediction is generated. The results and/or related information may be communicated to the subject by the subject's treating physician. Alternatively, the results may be communicated directly to a test subject by any means of communication, including writing, such as by providing a written report, electronic forms of communication, such as email, or telephone. Communication may be facilitated by use of a computer, such as in case of email communications. In certain embodiments, the communication containing results of a diagnostic test and/or conclusions drawn from and/or treatment recommendations based on the test, may be generated and delivered automatically to the subject using a combination of computer hardware and software which will be familiar to artisans skilled in telecommunications. One example of a healthcare-oriented communications system is described in U.S. Pat. No. 6,283,761; however, the present disclosure is not limited to methods which utilize this particular communications system. In certain embodiments of the methods of the disclosure, all or some of the method steps, including the assaying of samples, diagnosing of diseases, and communicating of assay results or diagnoses, may be carried out in diverse (e.g., foreign) jurisdictions.

In several embodiments, identification of a subject as having an Aspergillus species-associated condition results in the physician treating the subject, such as prescribing one or more therapeutic agents for inhibiting or delaying one or more signs and symptoms associated with the Aspergillus species-associated disorder/condition. In additional embodiments, the dose or dosing regimen is modified based on the information obtained using the methods disclosed herein.

The subject can be monitored while undergoing treatment using the methods described herein in order to assess the efficacy of the treatment protocol. In this manner, the length of time or the amount given to the subject can be modified based on the results obtained using the methods disclosed herein. The subject can also be monitored after the treatment using the methods described herein to monitor for relapse. In this manner, whether to resume treatment can be decided based on the results obtained using the methods disclosed herein.

### IV. Immunoassays for Diagnosing and Monitoring Aspergillus Species-Associated Conditions

The methods disclosed herein can be performed in the form of various immunoassay formats, which are well known in the art. There are two main types of immunoassays, homogeneous and heterogeneous. In homogeneous immunoassays, both the immunological reaction between an antigen and an antibody and the detection are carried out in a homogeneous reaction. Heterogeneous immunoassays include at least one separation step, which allows the differentiation of reaction products from unreacted reagents. A variety of immunoassays can be used to detect one or more of the molecules capable of detecting an Aspergillus species, such as Af, including detecting extracellular polysaccharides. In one example, one or more antigens associated with an Aspergillus species-associated disorder/condition are measured to diagnose an Af-associated disorder, such as aspergillosis, including IA. For example, one or more Af protein antigens listed in Table 1 are detected with a disclosed immunoassay. In one example, at least one or more of the following antigens are detected: Aspergillus catalase (Protein ID No. 70986104), GPI-anchored cell wall β-1,3-endogluconase (Eglc) (Protein ID No. 70985687), GPI-anchored cell wall organization protein (Ecm33) (Protein ID No. 70994734), Chr-like protein (Protein ID No. 27372089), Thioreduxin reductase (Protein ID No. 70992029), Peptidyl-prolyl cis-trans isomerase (Protein ID No. 70989309), Major allergen (Protein ID No. 83300352), Conserved hypothetical protein (Protein ID No. 159122886), Conserved hypothetical protein (Protein ID No. 70995516), 1,3-β-glucanosyltransferase (70989629), Adenoside deaminase (Protein ID No. 146323525), L-amino acid oxidase Lao (Protein ID No. 70986680), Glu/Leu/Phe/Val Dehydrogenase (70994774), Adenosyl homocysteinase (Protein ID No. 70995231), and Pigment biosynthesis protein (Protein ID No. 211909651).

In some examples, the disclosed immunoassay includes at least one, such as two, three, four, five, six, seven, eight, nine, ten, eleven, or more molecules associated with an Aspergillus species-associated condition or disease, such as an Af-associated condition or disease. In one example, the immunoassay includes at least one, such as two, three, four, five, six, seven, eight, nine, ten, or eleven molecules listed in Table 1. In one example, the assay includes at least Catalase, GPI-anchored cell wall β-1,3-endogluconase (Eglc), GPI-anchored cell wall organization protein (Ecm33), Chr-like protein, and/or Thioreduxin reductase.

ELISA is a heterogeneous immunoassay, which has been widely used in laboratory practice since the early 1970s, and can be used in the methods disclosed herein. The assay can be used to detect protein antigens in various formats. In the “sandwich” format the antigen being assayed is held between two different antibodies. In this method, a solid surface is first coated with a solid phase antibody. The test sample, containing the antigen (e.g., a diagnostic protein), or a composition containing the antigen, such as a urine sample from a subject of interest, is then added and the antigen is allowed to react with the bound antibody. Any unbound antigen is washed away. A known amount of enzyme-labeled antibody is then allowed to react with the bound antigen. Any excess unbound enzyme-linked antibody is washed away after the reaction. The substrate for the enzyme used in the assay is then added and the reaction between the substrate and the enzyme produces a color change. The amount of visual color change is a direct measurement of specific enzyme-conjugated bound antibody, and consequently the antigen present in the sample tested.

ELISA can also be used as a competitive assay. In the competitive assay format, the test specimen containing the antigen to be determined is mixed with a precise amount of enzyme-labeled antigen and both compete for binding to an anti-antigen antibody attached to a solid surface. Excess free enzyme-labeled antigen is washed off before the substrate for the enzyme is added. The amount of color intensity resulting from the enzyme-substrate interaction is a measure of the amount of antigen in the sample tested. A heterogeneous immunoassay, such as an ELISA, can be used to detect any molecules associated with an Aspergillus species, such Af.

In another example, immuno-PCR can be used to detect any of the molecules associated with an Aspergillus species, such as Af. Immuno-PCR is a modification of the conventional ELISA format in which the detecting antibody is labeled with a DNA label, and is applicable to the analysis of biological samples (see, e.g., U.S. Pat. No. 5,665,539 and U.S. Patent Application Publication No. 2005/0239108; all herein incorporated by reference). The amplification ability of PCR provides large amounts of the DNA label which can be detected by various methods, typically gel electrophoresis with conventional staining (e.g., Sano et al., Science, 258:120-122, 1992). This method can also include the direct conjugation of the DNA label to the antibody and replacement of gel electrophoresis by using labeled primers to generate a PCR product that can be assayed by ELISA or using real time quantitative PCR. In an example of the real-time PCR method, PCR is used to amplify DNA in a sample in the presence of a nonextendable dual labeled fluorogenic hybridization probe. One fluorescent dye serves as a reporter and its emission spectra is quenched by the second fluorescent dye. The method uses the 5′ nuclease activity of Taq polymerase to cleave a hybridization probe during the extension phase of PCR. The nuclease degradation of the hybridization probe releases the quenching of the reporter dye resulting in an increase in peak emission from the reporter. The reactions are monitored in real time.

Homogeneous immunoassays include, for example, the Enzyme Multiplied Immunoassay Technique (EMIT), which typically includes a biological sample comprising the biomarkers to be measured, enzyme-labeled molecules of the biomarkers to be measured, specific antibody or antibodies binding the biomarkers to be measured, and a specific enzyme chromogenic substrate. In a typical EMIT, excess of specific antibodies is added to a biological sample. If the biological sample contains the molecules to be detected, such molecules bind to the antibodies. A measured amount of the corresponding enzyme-labeled molecules is then added to the mixture. Antibody binding sites not occupied by molecules of the protein in the sample are occupied with molecules of the added enzyme-labeled protein. As a result, enzyme activity is reduced because only free enzyme-labeled protein can act on the substrate. The amount of substrate converted from a colorless to a colored form determines the amount of free enzyme left in the mixture. A high concentration of the protein to be detected in the sample causes higher absorbance readings. Less protein in the sample results in less enzyme activity and consequently lower absorbance readings. Inactivation of the enzyme label when the antigen-enzyme complex is antibody-bound makes the EMIT a useful system, enabling the test to be performed without a separation of bound from unbound compounds as is necessary with other immunoassay methods. A homogenous immunoassay, such as an EMIT, can be used to detect any of the molecules associated with an Aspergillus species-associated condition or disease, such as Af protein antigens listed in Table 1.

Immunoassay kits are also disclosed herein. These kits include, in separate containers (a) monoclonal antibodies having binding specificity for the polypeptides used in the diagnosis of an Aspergillus species-associated condition/disorder, such as an Af-associated condition/disorder; and (b) and anti-antibody immunoglobulins. This immunoassay kit may be utilized for the practice of the various methods provided herein. The monoclonal antibodies and the anti-antibody immunoglobulins can be provided in an amount of about 0.001 mg to 100 grams, and more preferably about 0.01 mg to 1 gram. The anti-antibody immunoglobulin may also be a polyclonal immunoglobulin, protein A or protein G or functional fragments thereof, which may be labeled prior to use by methods known in the art. In several embodiments, the immunoassay kit includes one, two, three or four or more antibodies that specifically bind to molecules associated with an Aspergillus species-associated condition or disease, such as Af protein antigens listed in Table 1. The immunoassay kit can also include one or more antibodies that specifically bind to one or more of these molecules. Thus, the kits can be used to detect one or more different molecules associated an Aspergillus species and thus, an Aspergillus species-associated condition, such as aspergillosis, including, but not limited to IA.

Immunoassays for polysaccharides and proteins differ in that a single antibody is used for both the capture and indicator roles for polysaccharides due to the presence of repeating epitopes. In contrast, two antibodies specific for distinct epitopes are required for immunoassay of proteins. Exemplary samples include biological samples obtained from subjects including, but not limited to, serum, blood and urine samples. In some examples, an exemplary sample includes bronchoalveolar lavage fluid.

In one particular example, a quantitative ELISA is constructed for detection of at least one of the Af protein antigens listed in Table 1. These immunoassays utilize antibodies, such as mAbs commercially available. Since a polysaccharide is a polyvalent repeating structure, a single mAb may be used for both the capture and indicator phases of an immunoassay. The only requirement is that the mAb have a sufficient affinity. A mAb with an affinity of about 0.5 μM has sufficient affinity.

### V. Capture Device Methods

The disclosed methods can be carried out using a sample capture device, such as a lateral flow device (for example a lateral flow test strip) that allows detection of one or more molecules, such as those described herein.

Point-of-use analytical tests have been developed for the routine identification or monitoring of health-related conditions (such as pregnancy, cancer, endocrine disorders, infectious diseases or drug abuse) using a variety of biological samples (such as urine, serum, plasma, blood, saliva). Some of the point-of-use assays are based on highly specific interactions between specific binding pairs, such as antigen/antibody, hapten/antibody, lectin/carbohydrate, apoprotein/cofactor and biotin/(strept)avidin. The assays are often performed with test strips in which a specific binding pair member is attached to a mobilizable material (such as a metal sol or beads made of latex or glass) or an immobile substrate (such as glass fibers, cellulose strips or nitrocellulose membranes). Particular examples of some of these assays are shown in U.S. Pat. Nos. 4,703,017; 4,743,560; and 5,073,484 (incorporated herein by reference). The test strips include a flow path from an upstream sample application area to a test site. For example, the flow path can be from a sample application area through a mobilization zone to a capture zone. The mobilization zone may contain a mobilizable marker that interacts with an analyte or analyte analog, and the capture zone contains a reagent that binds the analyte or analyte analog to detect the presence of an analyte in the sample.

Examples of migration assay devices, which usually incorporate within them reagents that have been attached to colored labels, thereby permitting visible detection of the assay results without addition of further substances are found, for example, in U.S. Pat. No. 4,770,853; WO 88/08534; and EP-A 0 299 428 (incorporated herein by reference). There are a number of commercially available lateral-flow type tests and patents disclosing methods for the detection of large analytes (MW greater than 1,000 Daltons) as the analyte flows through multiple zones on a test strip. Examples are found in U.S. Pat. No. 5,229,073 (measuring plasma lipoprotein levels), and U.S. Pat. Nos. 5,591,645; 4,168,146; 4,366,241; 4,855,240; 4,861,711; 5,120,643; European Patent No. 0296724; WO 97/06439; WO 98/36278; and WO 08/030546 (each of which are herein incorporated by reference). Multiple zone lateral flow test strips are disclosed in U.S. Pat. No. 5,451,504, U.S. Pat. No. 5,451,507, and U.S. Pat. No. 5,798,273 (incorporated by reference herein). U.S. Pat. No. 6,656,744 (incorporated by reference) discloses a lateral flow test strip in which a label binds to an antibody through a streptavidin-biotin interaction.

In particular examples, the methods disclosed herein include application of a biological sample (such as serum, whole blood or urine) from a test subject to a lateral flow test device for the detection of one or more molecules (such as one or more molecules associated with an Aspergillus species, such as Af, for example, combinations of molecules as described above) in the sample. The lateral flow test device includes one or more antibodies (such as antibodies that bind one or more of the molecules associated with an Aspergillus species, such as Af) at an addressable location. In a particular example, the lateral flow test device includes antibodies that bind at least one Af protein antigen listed in Table 1. The addressable locations can be, for example, a linear array or other geometric pattern that provides diagnostic information to the user. The binding of one or more molecules in the sample to the antibodies present in the test device is detected and the presence or amount of one or more molecules in the sample of the test subject is compared to a control, wherein a change in the presence or amount of one or more molecules in the sample from the test subject as compared to the control indicates that the subject has an Af-associated condition, such as aspergillosis, including IA.

Devices described herein generally include a strip of absorbent material (such as a microporous membrane), which, in some instances, can be made of different substances each joined to the other in zones, which may be abutted and/or overlapped. In some examples, the absorbent strip can be fixed on a supporting non-interactive material (such as nonwoven polyester), for example, to provide increased rigidity to the strip. Zones within each strip may differentially contain the specific binding partner(s) and/or other reagents required for the detection and/or quantification of the particular analyte being tested for, for example, one or more molecules disclosed herein. Thus these zones can be viewed as functional sectors or functional regions within the test device.

In general, a fluid sample is introduced to the strip at the proximal end of the strip, for instance by dipping or spotting. A sample is collected or obtained using methods well known to those skilled in the art. The sample containing the particular molecules to be detected may be obtained from any biological source. Examples of biological sources include blood serum, blood plasma, urine, BALF, spinal fluid, saliva, fermentation fluid, lymph fluid, tissue culture fluid and ascites fluid of a human or animal. In a particular example, the biological source is saliva. In one particular example, the biological source is whole blood, such as a sample obtained from a finger prick. The sample may be diluted, purified, concentrated, filtered, dissolved, suspended or otherwise manipulated prior to assay to optimize the immunoassay results. The fluid migrates distally through all the functional regions of the strip. The final distribution of the fluid in the individual functional regions depends on the adsorptive capacity and the dimensions of the materials used.

Another common feature to be considered in the use of assay devices is a means to detect the formation of a complex between an analyte (such as one or more molecules described herein) and a capture reagent (such as one or more antibodies). A detector (also referred to as detector reagent) serves this purpose. A detector may be integrated into an assay device (for example included in a conjugate pad, as described below), or may be applied to the device from an external source.

A detector may be a single reagent or a series of reagents that collectively serve the detection purpose. In some instances, a detector reagent is a labeled binding partner specific for the analyte (such as a gold-conjugated antibody for a particular protein of interest, for example those described herein).

In other instances, a detector reagent collectively includes an unlabeled first binding partner specific for the analyte and a labeled second binding partner specific for the first binding partner and so forth. Thus, the detector can be a labeled antibody specific for a protein described herein. The detector can also be an unlabeled first antibody specific for the protein of interest and a labeled second antibody that specifically binds the unlabeled first antibody. In each instance, a detector reagent specifically detects bound analyte of an analyte-capture reagent complex and, therefore, a detector reagent preferably does not substantially bind to or react with the capture reagent or other components localized in the analyte capture area. Such non-specific binding or reaction of a detector may provide a false positive result. Optionally, a detector reagent can specifically recognize a positive control molecule (such as a non-specific human IgG for a labeled Protein A detector, or a labeled Protein G detector, or a labeled anti-human Ab(Fc)) that is present in a secondary capture area.

### Flow-Through Device Construction and Design

Representative flow-through assay devices are described in U.S. Pat. Nos. 4,246,339; 4,277,560; 4,632,901; 4,812,293; 4,920,046; and 5,279,935; U.S. Patent Application Publication Nos. 20030049857 and 20040241876; and WO 08/030546. A flow-through device involves a capture reagent (such as one or more antibodies) immobilized on a solid support, typically, a membrane (such as, nitrocellulose, nylon, or PVDF). Characteristics of useful membranes have been previously described; however, it is useful to note that in a flow-through assay capillary rise is not a particularly important feature of a membrane as the sample moves vertically through the membrane rather than across it as in a lateral flow assay. In a simple representative format, the membrane of a flow-through device is placed in functional or physical contact with an absorbent layer (see, e.g., description of “absorbent pad” below), which acts as a reservoir to draw a fluid sample through the membrane. Optionally, following immobilization of a capture reagent, any remaining protein-binding sites on the membrane can be blocked (either before or concurrent with sample administration) to minimize nonspecific interactions.

In operation of a flow-through device, a fluid sample (such as a bodily fluid sample) is placed in contact with the membrane. Typically, a flow-through device also includes a sample application area (or reservoir) to receive and temporarily retain a fluid sample of a desired volume. The sample passes through the membrane matrix. In this process, an analyte in the sample (such as one or more protein, for example, one or more molecules described herein) can specifically bind to the immobilized capture reagent (such as one or more antibodies). Where detection of an analyte-capture reagent complex is desired, a detector reagent (such as labeled antibodies that specifically bind one or more molecules) can be added with the sample or a solution containing a detector reagent can be added subsequent to application of the sample. If an analyte is specifically bound by capture reagent, a visual representative attributable to the particular detector reagent can be observed on the surface of the membrane. Optional wash steps can be added at any time in the process, for instance, following application of the sample, and/or following application of a detector reagent.

### Lateral Flow Device Construction and Design

Lateral flow devices are commonly known in the art. Briefly, a lateral flow device is an analytical device having as its essence a test strip, through which flows a test sample fluid that is suspected of containing an analyte of interest. The test fluid and any suspended analyte can flow along the strip to a detection zone in which the analyte (if present) interacts with a capture agent and a detection agent to indicate a presence, absence and/or quantity of the analyte.

Numerous lateral flow analytical devices have been disclosed, and include those shown in U.S. Pat. Nos. 4,168,146; 4,313,734; 4,366,241; 4,435,504; 4,775,636; 4,703,017; 4,740,468; 4,806,311; 4,806,312; 4,861,711; 4,855,240; 4,857,453; 4,861,711; 4,943,522; 4,945,042; 4,496,654; 5,001,049; 5,075,078; 5,126,241; 5,120,643; 5,451,504; 5,424,193; 5,712,172; 6,555,390; 6,258,548; 6,699,722; 6,368,876 and 7,517,699; EP 0810436; EP 0296724; WO 92/12428; WO 94/01775; WO 95/16207; WO 97/06439; WO 98/36278; and WO 08/030546, each of which is incorporated by reference. Further, there are a number of commercially available lateral flow type tests and patents disclosing methods for the detection of large analytes (MW greater than 1,000 Daltons). U.S. Pat. No. 5,229,073 describes a semiquantitative competitive immunoassay lateral flow method for measuring plasma lipoprotein levels. This method utilizes a plurality of capture zones or lines containing immobilized antibodies to bind both the labeled and free lipoprotein to give a semi-quantitative result. In addition, U.S. Pat. No. 5,591,645 provides a chromatographic test strip with at least two portions. The first portion includes a movable tracer and the second portion includes an immobilized binder capable of binding to the analyte.

Many lateral flow devices are one-step lateral flow assays in which a biological fluid is placed in a sample area on a bibulous strip (though non-bibulous materials can be used, and rendered bibulous, e.g., by applying a surfactant to the material), and allowed to migrate along the strip until the liquid comes into contact with a specific binding partner (such as an antibody) that interacts with an analyte (such as one or more molecules) in the liquid. Once the analyte interacts with the binding partner, a signal (such as a fluorescent or otherwise visible dye) indicates that the interaction has occurred. Multiple discrete binding partners (such as antibodies) can be placed on the strip (for example in parallel lines) to detect multiple analytes (such as two or more molecules) in the liquid. The test strips can also incorporate control indicators, which provide a signal that the test has adequately been performed, even if a positive signal indicating the presence (or absence) of an analyte is not seen on the strip.

The construction and design of lateral flow devices is very well known in the art, as described, for example, in Millipore Corporation, A Short Guide Developing Immunochromatographic Test Strips, 2nd Edition, pp. 1-40, 1999, available by request at (800) 645-5476; and Schleicher & Schuell, Easy to Work with BioScience, Products and Protocols 2003, pp. 73-98, 2003, 2003, available by request at Schleicher & Schuell BioScience, Inc., 10 Optical Avenue, Keene, N.H. 03431, (603) 352-3810; both of which are incorporated herein by reference.

Lateral flow devices have a wide variety of physical formats that are equally well known in the art. Any physical format that supports and/or houses the basic components of a lateral flow device in the proper function relationship is contemplated by this disclosure.

In some embodiments, the lateral flow strip is divided into a proximal sample application pad, an intermediate test result zone, and a distal absorbent pad. The flow strip is interrupted by a conjugate pad that contains labeled conjugate (such as gold- or latex-conjugated antibody specific for the target analyte or an analyte analog). A flow path along strip passes from proximal pad, through conjugate pad, into test result zone, for eventual collection in absorbent pad. Selective binding agents are positioned on a proximal test line in the test result membrane. A control line is provided in test result zone, slightly distal to the test line. For example, in a competitive assay, the binding agent in the test line specifically binds the target analyte, while the control line less specifically binds the target analyte.

In operation of the particular embodiment of a lateral flow device, a fluid sample containing an analyte of interest, such as one or more molecules described herein (for example, Af protein antigens listed in Table 1, as discussed above), is applied to the sample pad. In some examples, the sample may be applied to the sample pad by dipping the end of the device containing the sample pad into the sample (such as serum or urine) or by applying the sample directly onto the sample pad (for example by placing the sample pad in the mouth of the subject). In other examples where a sample is whole blood, an optional developer fluid is added to the blood sample to cause hemolysis of the red blood cells and, in some cases, to make an appropriate dilution of the whole blood sample.

From the sample pad, the sample passes, for instance by capillary action, to the conjugate pad. In the conjugate pad, the analyte of interest, such as a protein of interest, may bind (or be bound by) a mobilized or mobilizable detector reagent, such as an antibody (such as antibody that recognizes one or more of the molecules described herein). For example, a protein analyte may bind to a labeled (e.g., gold-conjugated or colored latex particle-conjugated) antibody contained in the conjugate pad. The analyte complexed with the detector reagent may subsequently flow to the test result zone where the complex may further interact with an analyte-specific binding partner (such as an antibody that binds a particular protein, an anti-hapten antibody, or streptavidin), which is immobilized at the proximal test line. In some examples, a protein complexed with a detector reagent (such as gold-conjugated antibody) may further bind to unlabeled, oxidized antibodies immobilized at the proximal test line. The formation of a complex, which results from the accumulation of the label (e.g., gold or colored latex) in the localized region of the proximal test line is detected. The control line may contain an immobilized, detector-reagent-specific binding partner, which can bind the detector reagent in the presence or absence of the analyte. Such binding at the control line indicates proper performance of the test, even in the absence of the analyte of interest. The test results may be visualized directly, or may measured using a reader (such as a scanner). The reader device may detect color or fluorescence from the readout area (for example, the test line and/or control line).

In another embodiment of a lateral flow device, there may be a second (or third, fourth, or more) test line located parallel or perpendicular (or in any other spatial relationship) to test line in test result zone. The operation of this particular embodiment is similar to that described in the immediately preceding paragraph with the additional considerations that (i) a second detector reagent specific for a second analyte, such as another antibody, may also be contained in the conjugate pad, and (ii) the second test line will contain a second specific binding partner having affinity for a second analyte, such as a second protein in the sample. Similarly, if a third (or more) test line is included, the test line will contain a third (or more) specific binding partner having affinity for a third (or more) analyte.

1. Sample Pad

The sample pad is a component of a lateral flow device that initially receives the sample, and may serve to remove particulates from the sample. Among the various materials that may be used to construct a sample pad (such as glass fiber, woven fibers, screen, non-woven fibers, cellosic fibers or paper), a cellulose sample pad may be beneficial if a large bed volume (e.g., 250 μl/cm²) is a factor in a particular application. Sample pads may be treated with one or more release agents, such as buffers, salts, proteins, detergents, and surfactants. Such release agents may be useful, for example, to promote resolubilization of conjugate-pad constituents, and to block non-specific binding sites in other components of a lateral flow device, such as a nitrocellulose membrane. Representative release agents include, for example, trehalose or glucose (1%-5%), PVP or PVA (0.5%-2%), Tween 20 or Triton X-100 (0.1%-1%), casein (1%-2%), SDS (0.02%-5%), and PEG (0.02%-5%).

2. Membrane and Application Solution:

The types of membranes useful in a lateral flow device (such as nitrocellulose (including pure nitrocellulose and modified nitrocellulose), nitrocellulose direct cast on polyester support, polyvinylidene fluoride, or nylon), and considerations for applying a capture reagent to such membranes have been discussed previously.

In some embodiments, membranes comprising nitrocellulose are preferably in the form of sheets or strips. The thickness of such sheets or strips may vary within wide limits, for example, from about 0.01 to 0.5 mm, from about 0.02 to 0.45 mm, from about 0.05 to 0.3 mm, from about 0.075 to 0.25 mm, from about 0.1 to 0.2 mm, or from about 0.11 to 0.15 mm. The pore size of such sheets or strips may similarly vary within wide limits, for example from about 0.025 to 15 microns, or more specifically from about 0.1 to 3 microns; however, pore size is not intended to be a limiting factor in selection of the solid support. The flow rate of a solid support, where applicable, can also vary within wide limits, for example from about 12.5 to 90 sec/cm (i.e., 50 to 300 sec/4 cm), about 22.5 to 62.5 sec/cm (i.e., 90 to 250 sec/4 cm), about 25 to 62.5 sec/cm (i.e., 100 to 250 sec/4 cm), about 37.5 to 62.5 sec/cm (i.e., 150 to 250 sec/4 cm), or about 50 to 62.5 sec/cm (i.e., 200 to 250 sec/4 cm). In specific embodiments of devices described herein, the flow rate is about 62.5 sec/cm (i.e., 250 sec/4 cm). In other specific embodiments of devices described herein, the flow rate is about 37.5 sec/cm (i.e., 150 sec/4 cm).

3. Conjugate Pad

The conjugate pad serves to, among other things, hold a detector reagent. Suitable materials for the conjugate pad include glass fiber, polyester, paper, or surface modified polypropylene. In some embodiments, a detector reagent may be applied externally, for example, from a developer bottle, in which case a lateral flow device need not contain a conjugate pad (see, for example, U.S. Pat. No. 4,740,468).

Detector reagent(s) contained in a conjugate pad is typically released into solution upon application of the test sample. A conjugate pad may be treated with various substances to influence release of the detector reagent into solution. For example, the conjugate pad may be treated with PVA or PVP (0.5% to 2%) and/or Triton X-100 (0.5%). Other release agents include, without limitation, hydroxypropylmethyl cellulose, SDS, Brij and β-lactose. A mixture of two or more release agents may be used in any given application. In a particular disclosed embodiment, the detector reagent in conjugate pad is a gold-conjugated antibody.

4. Absorbent Pad

The use of an absorbent pad in a lateral flow device is optional. The absorbent pad acts to increase the total volume of sample that enters the device. This increased volume can be useful, for example, to wash away unbound analyte from the membrane. Any of a variety of materials is useful to prepare an absorbent pad, for example, cellulosic filters or paper. In some device embodiments, an absorbent pad can be paper (i.e., cellulosic fibers). One of skill in the art may select a paper absorbent pad on the basis of, for example, its thickness, compressibility, manufacturability, and uniformity of bed volume. The volume uptake of an absorbent made may be adjusted by changing the dimensions (usually the length) of an absorbent pad.

### VI. Methods for Inducing an Immune Response

Methods of inducing an immune response to an Aspergillus species-associated condition are also disclosed. The methods include the use of the immunogenic Aspergillus species polypeptides, such as Af polypeptides disclosed herein, nucleic acids encode these polypeptides, and/or viral vectors encoding an immunogenic Af polypeptide, alone or in conjunction with other agents, such as traditional Af-associated condition therapies, including traditional therapies for aspergillosis, including IA. In several embodiments, the methods include administering to a subject with an Aspergillus species-associated condition, such as an Af-associated condition, a therapeutically effective amount of one or more Aspergillus species polypeptides, such as those disclosed herein, in order to generate an immune response.

The methods can include selecting a subject in need of treatment, such as a subject having or at risk acquiring Af. In exemplary applications, compositions are administered to a subject having a disease, such as an Aspergillus species-associated condition (e.g., IA), in an amount sufficient to raise an immune response to Aspergillus species-associated antigen-expressing cells. Administration induces a sufficient immune response to slow the proliferation of such cells or to inhibit their growth, or to reduce a sign or a symptom of the Aspergillus species-associated condition. Amounts effective for this use will depend upon the severity of the disease, the general state of the patient's health, and the robustness of the patient's immune system. In one example, a therapeutically effective amount of the compound is that which provides either subjective relief of a symptom(s) or an objectively identifiable improvement as noted by the clinician or other qualified observer.

An Aspergillus species polypeptide, such as an Af polypeptide, can be administered by any means known to one of skill in the art (see Banga, A., “Parenteral Controlled Delivery of Therapeutic Peptides and Proteins,” in Therapeutic Peptides and Proteins, Technomic Publishing Co., Inc., Lancaster, Pa., 1995) either locally or systemically, such as by intramuscular, subcutaneous, intraperitoneal or intravenous injection, but even oral, nasal, transdermal or anal administration is contemplated. In one embodiment, administration is by subcutaneous or intramuscular injection. To extend the time during which the peptide or protein is available to stimulate a response, the peptide or protein can be provided as an implant, an oily injection, or as a particulate system. The particulate system can be a microparticle, a microcapsule, a microsphere, a nanocapsule, or similar particle. (see, e.g., Banga, supra). A particulate carrier based on a synthetic polymer has been shown to act as an adjuvant to enhance the immune response, in addition to providing a controlled release. Aluminum salts can also be used as adjuvants to produce an immune response.

In one specific, non-limiting example, the Aspergillus species polypeptide, such as the Af polypeptide, is administered in a manner to direct the immune response to a cellular response (that is, a cytotoxic T lymphocyte (CTL) response), rather than a humoral (antibody) response.

Optionally, one or more cytokines, such as IL-2, IL-6, IL-12, RANTES, GM-CSF, TNF-α, or IFN-γ, one or more growth factors, such as GM-CSF or G-CSF, one or more costimulatory molecules, such as ICAM-1, LFA-3, CD72, B7-1, B7-2, or other B7 related molecules; one or more molecules such as OX-40L or 41 BBL, or combinations of these molecules, can be used as biological adjuvants (see, for example, Salgaller et al., 1998, J. Surg. Oncol. 68(2):122-38; Lotze et al., 2000, Cancer J Sci. Am. 6(Suppl 1):S61-6; Cao et al., 1998, Stem Cells 16(Suppl 1):251-60; Kuiper et al., 2000, Adv. Exp. Med. Biol. 465:381-90). These molecules can be administered systemically (or locally) to the host. In several examples, IL-2, RANTES, GM-CSF, TNF-α, IFN-γ, G-CSF, LFA-3, CD72, B7-1, B7-2, B7-1, B7-2, OX-40L, 41 BBL and ICAM-1 are administered.

A number of means for inducing cellular responses, both in vitro and in vivo, are known. Lipids have been identified as agents capable of assisting in priming CTL in vivo against various antigens. For example, as described in U.S. Pat. No. 5,662,907, palmitic acid residues can be attached to the alpha and epsilon amino groups of a lysine residue and then linked (for example, via one or more linking residues, such as glycine, glycine-glycine, serine, serine-serine, or the like) to an immunogenic peptide. The lipidated peptide can then be injected directly in a micellar form, incorporated in a liposome, or emulsified in an adjuvant. Further, as the induction of neutralizing antibodies can also be primed with the same molecule conjugated to a peptide which displays an appropriate epitope, two compositions can be combined to elicit both humoral and cell-mediated responses where that is deemed desirable.

In yet another embodiment, to induce a CTL response to an immunogenic Aspergillus species polypeptide, such as an Af polypeptide, a MHC Class II-restricted T-helper epitope is added to the immunogenic polypeptide to induce T-helper cells to secrete cytokines in the microenvironment to activate CTL precursor cells. The technique further involves adding short lipid molecules to retain the construct at the site of the injection for several days to localize the antigen at the site of the injection and enhance its proximity to dendritic cells or other “professional” antigen presenting cells over a period of time (see Chesnut et al., “Design and Testing of Peptide-Based Cytotoxic T-Cell-Mediated Immunotherapeutics to Treat Infectious Diseases and Cancer,” in Powell et al., eds., Vaccine Design, the Subunit and Adjuvant Approach, Plenum Press, New York, 1995).

A pharmaceutical composition including a disclosed polypeptide is thus provided. These compositions are use to generate an immune response, such as for immunotherapy. In one embodiment, a disclosed polypeptide is mixed with an adjuvant containing two or more of a stabilizing detergent, a micelle-forming agent, and an oil. Suitable stabilizing detergents, micelle-forming agents, and oils are detailed in U.S. Pat. No. 5,585,103; U.S. Pat. No. 5,709,860; U.S. Pat. No. 5,270,202; and U.S. Pat. No. 5,695,770, all of which are incorporated by reference. A stabilizing detergent is any detergent that allows the components of the emulsion to remain as a stable emulsion. Such detergents include polysorbate, 80 (TWEEN) (Sorbitan-mono-9-octadecenoate-poly(oxy-1,2-ethanediyl; manufactured by ICI Americas, Wilmington, Del.), TWEEN 40™, TWEEN 20™, TWEEN 60™, Zwittergent™ 3-12, TEEPOL HB7™, and SPAN 85™. These detergents are usually provided in an amount of approximately 0.05 to 0.5%, such as at about 0.2%. A micelle forming agent is an agent which is able to stabilize the emulsion formed with the other components such that a micelle-like structure is formed. Such agents generally cause some irritation at the site of injection in order to recruit macrophages to enhance the cellular response. Examples of such agents include polymer surfactants described by BASF Wyandotte publications, e.g., Schmolka, J. Am. Oil. Chem. Soc. 54:110, 1977, and Hunter et al., J. Immunol 129:1244, 1981, PLURONIC™ L62LF, L101, and L64, PEG1000, and TETRONIC™ 1501, 150 R1, 701, 901, 1301, and 130R1. The chemical structures of such agents are well known in the art. In one embodiment, the agent is chosen to have a hydrophile-lipophile balance (HLB) of between 0 and 2, as defined by Hunter and Bennett, J. Immun. 133:3167, 1984. The agent can be provided in an effective amount, for example between 0.5 and 10%, or in an amount between 1.25 and 5%.

The oil included in the composition is chosen to promote the retention of the antigen in oil-in-water emulsion, such as to provide a vehicle for the desired antigen, and preferably has a melting temperature of less than 65° C. such that emulsion is formed either at room temperature (about 20° C. to 25° C.), or once the temperature of the emulsion is brought down to room temperature. Examples of such oils include squalene, Squalane, EICOSANE™, tetratetracontane, glycerol, and peanut oil or other vegetable oils. In one specific, non-limiting example, the oil is provided in an amount between 1 and 10%, or between 2.5 and 5%. The oil should be both biodegradable and biocompatible so that the body can break down the oil over time, and so that no adverse affects, such as granulomas, are evident upon use of the oil.

In one embodiment, the adjuvant is a mixture of stabilizing detergents, micelle-forming agent, and oil available under the name PROVAX® (IDEC Pharmaceuticals, San Diego, Calif.). An adjuvant can also be an immunostimulatory nucleic acid, such as a nucleic acid including a CpG motif, or a biological adjuvant (see above).

Controlled release parenteral formulations can be made as implants, oily injections, or as particulate systems. For a broad overview of protein delivery systems, see Banga, Therapeutic Peptides and Proteins: Formulation, Processing, and Delivery Systems, Technomic Publishing Company, Inc., Lancaster, Pa., 1995. Particulate systems include microspheres, microparticles, microcapsules, nanocapsules, nanospheres, and nanoparticles. Microcapsules contain the therapeutic protein as a central core. In microspheres, the therapeutic agent is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly (see Kreuter, Colloidal Drug Delivery Systems, J. Kreuter, ed., Marcel Dekker, Inc., New York, N.Y., pp. 219-342, 1994; Tice & Tabibi, Treatise on Controlled Drug Delivery, A. Kydonieus, ed., Marcel Dekker, Inc. New York, N.Y., pp. 315-339, 1992).

Polymers can be used for ion-controlled release. Various degradable and nondegradable polymeric matrices for use in controlled drug delivery are known in the art (Langer, Accounts Chem. Res. 26:537, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; and Pec, J. Parent. Sci. Tech. 44(2):58, 1990). Alternatively, hydroxyapatite has been used as a microcarrier for controlled release of proteins (Ijntema et al., Int. J. Pharm. 112:215, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa., 1993). Numerous additional systems for controlled delivery of therapeutic proteins are known (e.g., U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; and U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342; and U.S. Pat. No. 5,534,496).

In another embodiment, a pharmaceutical composition includes a nucleic acid encoding an Aspergillus species-associated polypeptide, such as an Af-associated polypeptide. A therapeutically effective amount of the polynucleotide can be administered to a subject in order to generate an immune response. In one specific, non-limiting example, a therapeutically effective amount of the polynucleotide is administered to a subject to treat one or more signs and symptoms associated with the Aspergillus species-associated condition, such as the Af-associated condition.

Optionally, one or more cytokines, such as IL-2, IL-6, IL-12, RANTES, GM-CSF, TNF-α, or IFN-γ, one or more growth factors, such as GM-CSF or G-CSF, one or more costimulatory molecules, such as ICAM-1, LFA-3, CD72, B7-1, B7-2, or other B7 related molecules; one or more molecules such as OX-40L or 41 BBL, or combinations of these molecules, can be used as biological adjuvants (see, for example, Salgaller et al., 1998, J. Surg. Oncol. 68(2):122-38; Lotze et al., 2000, Cancer J Sci. Am. 6(Suppl 1):S61-6; Cao et al., 1998, Stem Cells 16(Suppl 1):251-60; Kuiper et al., 2000, Adv. Exp. Med. Biol. 465:381-90). These molecules can be administered systemically to the host. It should be noted that these molecules can be co-administered via insertion of a nucleic acid encoding the molecules into a vector, for example, a recombinant pox vector (see, for example, U.S. Pat. No. 6,045,802). In various embodiments, the nucleic acid encoding the biological adjuvant can be cloned into same vector as the Aspergillus species-associated polypeptide coding sequences, such as the Af-associated polypeptide coding sequence, or the nucleic acid can be cloned into one or more separate vectors for co-administration.

One approach to administration of nucleic acids is direct immunization with plasmid DNA, such as with a mammalian expression plasmid. The nucleotide sequence encoding an Aspergillus species-associated polypeptide can be placed under the control of a promoter to increase expression of the molecule.

Immunization by nucleic acid constructs is well known in the art and taught, for example, in U.S. Pat. No. 5,643,578 (which describes methods of immunizing vertebrates by introducing DNA encoding a desired antigen to elicit a cell-mediated or a humoral response), and U.S. Pat. No. 5,593,972 and U.S. Pat. No. 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™, negatively charged cage-like structures of 30-40 nm in size formed spontaneously on mixing cholesterol and Quil A™ (saponin). Protective immunity has been generated in a variety of experimental models of infection, including toxoplasmosis and Epstein-Barr virus-induced tumors, using ISCOMS™ as the delivery vehicle for antigens (Mowat and Donachie, Immunol. Today 12:383, 1991). Doses of antigen as low as 1 μg encapsulated in ISCOMS™ have been found to produce Class I mediated CTL responses (Takahashi et al., Nature 344:873, 1990).

In another approach to using nucleic acids for immunization, a polypeptide can also be expressed by attenuated viral hosts or vectors or bacterial vectors. Recombinant vaccinia virus, adeno-associated virus (AAV), herpes virus, retrovirus, or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991).

A first recombinant virus, such as a poxvirus (for example, vaccine virus) encoding an Af-associated immunogenic polypeptide can be used in conjunction with a second recombinant virus which has incorporated into a viral genome or infectable portion thereof one or more genes or DNA sequences encoding B7-1, B7-2, or B7-1 and B7-2, wherein the composition is able to coinfect a host cell resulting in coexpression of the polypeptide and the B7-1, B7-2, or B7-1 and B7-2 encoding genes or DNA sequences (see U.S. Pat. No. 6,893,869, and U.S. Pat. No. 6,045,908, which are incorporated by reference herein).

When a viral vector is utilized, it is desirable to provide the recipient with a dosage of each recombinant virus in the composition in the range of from about 10⁵ to about 10¹⁰ plaque forming units/mg mammal, although a lower or higher dose can be administered. The composition of recombinant viral vectors can be introduced into a mammal either prior to any evidence of an Aspergillus species-associated condition, such as an Af-associated condition, or to mediate regression of the disease in a mammal afflicted with the Aspergillus species-associated condition, such as the Af-associated condition. Examples of methods for administering the composition into mammals include, but are not limited to, exposure of cells to the recombinant virus ex vivo, or injection of the composition into the affected tissue or intravenous, subcutaneous, intradermal or intramuscular administration of the virus. Generally, the quantity of recombinant viral vector, carrying the nucleic acid sequence of one or more Aspergillus species-associated polypeptides to be administered is based on the titer of virus particles. An exemplary range of the immunogen to be administered is 10⁵ to 10¹⁰ virus particles per mammal, such as a human.

In the embodiment where a combination of a first recombinant viral vector carrying a nucleic acid sequence of one or more Aspergillus species-associated polypeptides and a second recombinant viral vector carrying the nucleic acid sequence of one or more immunostimulatory molecules is used, the mammal can be immunized with different ratios of the first and second recombinant viral vector. In one embodiment the ratio of the first vector to the second vector is about 1:1, or about 1:3, or about 1:5. Optimal ratios of the first vector to the second vector may easily be titered using the methods known in the art (see, for example, U.S. Pat. No. 6,893,869, incorporated herein by reference).

In one embodiment the recombinant viruses have been constructed to express cytokines (such as TNF-α, IL-6, GM-CSF, and IL-2), and co-stimulatory and accessory molecules (B7-1, B7-2) alone and in a variety of combinations. Simultaneous production of an immunostimulatory molecule and the Aspergillus species-associated polypeptide enhances the generation of specific effectors. Without being bound by theory, dependent upon the specific immunostimulatory molecules, different mechanisms might be responsible for the enhanced immunogenicity: augmentation of help signal (IL-2), recruitment of professional APC (GM-CSF), increase in CTL frequency (IL-2), effect on antigen processing pathway and MHC expression (IFNγ and TNFα) and the like. For example, IL-2, IL-6, interferon, tumor necrosis factor, or a nucleic acid encoding these molecules, can be administered in conjunction with an Aspergillus species polypeptide, such as an Af immunogenic polypeptide, or a nucleic acid encoding such polypeptide. The co-expression of a disclosed polypeptide together with at least one immunostimulatory molecule can be effective in an animal model to show anti-Aspergillus species effects, such as anti-Af effects.

In one embodiment, a nucleic acid encoding an Af-associated polypeptide is introduced directly into cells. For example, the nucleic acid can be loaded onto gold microspheres by standard methods and introduced into the skin by a device such as Bio-Rad's Helios™ Gene Gun. The nucleic acids can be “naked,” consisting of plasmids under control of a strong promoter. Typically, the DNA is injected into muscle, although it can also be injected directly into other sites, including tissues in proximity to metastases. Dosages for injection are usually around 0.5 μg/kg to about 50 mg/kg, and typically are about 0.005 mg/kg to about 5 mg/kg (see, for example, U.S. Pat. No. 5,589,466).

In one specific, non-limiting example, a pharmaceutical composition for intravenous administration would include about 0.1 μg to 10 mg of an immunogenic Af polypeptide per patient per day. Dosages from 0.1 up to about 100 mg per patient per day can be used, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remingtons Pharmaceutical Sciences, 19^{th} Ed., Mack Publishing Company, Easton, Pa., 1995.

Single or multiple administrations of the compositions are administered depending on the dosage and frequency as required and tolerated by the subject. In one embodiment, the dosage is administered once as a bolus, but in another embodiment can be applied periodically until a therapeutic result is achieved. Generally, the dose is sufficient to treat or ameliorate symptoms or signs of disease without producing unacceptable toxicity to the subject. Systemic or local administration can be utilized.

In another method, antigen presenting cells (APCs), such as dendritic cells, are pulsed or co-incubated with peptides comprising an Aspergillus species polypeptide, such as an Af polypeptide, in vitro. In one specific, non-limiting example, the antigen presenting cells can be autologous cells. A therapeutically effective amount of the antigen presenting cells can then be administered to a subject.

The Aspergillus species polypeptide, such as the Af-associated polypeptide, can be delivered to the dendritic cells or to dendritic cell precursors via any method known in the art, including, but not limited to, pulsing dendritic cells directly with antigen, or utilizing a broad variety of antigen delivery vehicles, such as, for example, liposomes, or other vectors known to deliver antigen to cells. In one specific, non-limiting example an antigenic formulation includes about 0.1 μg to about 1,000 μg, or about 1 to about 100 μg of a selected polypeptide. The polypeptide can also be administered with agents that promote dendritic cell maturation. Specific, non-limiting examples of agents of use are interleukin-4 (IL-4) and granulocyte/macrophage colony stimulating factor (GM-CSF), or flt-3 ligand (flt-3L). The preparation can also contain buffers, excipients, and preservatives, amongst other ingredients.

In one embodiment, mature antigen presenting cells are generated to present the immunogenic polypeptide. These dendritic cells are then administered alone (or in combination with another agent) to a subject with an Aspergillus species-associated condition, such as aspergillosis, including IA.

Alternatively, the APCs are used to sensitize CD8 cells, such as peripheral blood lymphocytes (PBLs). The PBLs can be from the same subject (autologous) that is to be treated. Alternatively, the PBLs can be heterologous. However, they should at least be MHC Class-I restricted to the HLA types the subject possesses. An effective amount of the sensitized cells are then administered to the subject.

Peripheral blood mononuclear cells (PBMCs) can be used as the responder cell source of CTL precursors. The appropriate antigen-presenting cells are incubated with peptide, after which the peptide-loaded antigen-presenting cells are then incubated with the responder cell population under optimized culture conditions. Positive CTL activation can be determined by assaying the culture for the presence of CTLs that kill radio-labeled target cells, both specific peptide-pulsed targets as well as target cells expressing endogenously processed forms of the antigen from which the peptide sequence was derived, such as Af.

The cells can be administered to a subject to inhibit one or more activities associated with Aspergillus species, including Af, including growth of cells expressing one or more Aspergillus species-associated molecules (such as those included in Table 1).

In these applications, a therapeutically effective amount of activated antigen presenting cells, or activated lymphocytes, are administered to a subject suffering from a disease, in an amount sufficient to raise an immune response to Af-associated molecules expressing cells. The resulting immune response is sufficient to slow the proliferation of such cells or to inhibit their growth, or to reduce a sign or a symptom of the Af-associated condition or disease.

In a supplemental method, any of these immunotherapies is augmented by administering a cytokine, such as interleukin (IL)-2, IL-3, IL-6, IL-10, IL-12, IL-15, GM-CSF, or interferons.

In a further method, any of these immunotherapies is augmented by administering an agent, such as, but not limited to, agents traditionally used to treat one or more systems associated with Aspergillus species, Af.

i. Immunogenic Aspergillus Species Molecules Including Peptides

Exemplary immunogenic Aspergillus species molecules, such as Af molecules, including immunogenic Af peptides, include those that are identified herein as being associated with an Aspergillus species, such as Af.

In one example, an immunogenic “Aspergillus species peptide” is a series of contiguous amino acid residues from an Aspergillus species-associated protein, such as an Af-associated protein. In some examples, the immunogenic Af peptides are about 7 amino acids in length to about 250 amino acids in length, for example, the may be at least 7, 8, 9, 10 or 11 residues in length, for example 10-30 residues, or 10-21 residues. The immunogenic Af peptide can be incorporated into a fusion protein or the immunogenic Af peptide can be incorporated into a polymer of repeating Af peptides, or Af peptides joined by non-Af peptide linkers. The Af peptides in some examples are about or no more than about 7, about 8, about 9, about 10, about 11, about 12, about 13, about 14, about 15, about 16, about 17, about 18, about 19, about 20, about 21, about 22, about 23, as about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, about 200, about 201, about 202, about 203, about 204, about 205, about 206, about 207, about 208, about 209, about 210, about 211, about 212, about 213, about 214, about 215, about 216, about 217, about 218, about 219, about 220, about 221, about 222, about 223, about 224, about 225, about 226, about 227, about 228, about 229, about 230, about 231, about 232, about 233, about 234, about 235, about 236, about 237, about 238, about 239, about 240, about 241, about 242, about 243, about 244, about 245, about 246, about 247, about 248, about 249, or about 250 amino acids in length, for example about 8 to about 250, about 10 to about 150, about 12 to about 30, about 14 to about 20 amino acids in length or greater. In this context, it is understood that “about” refers to an integer quantity. In some examples, the Af peptide is even greater than 250 amino acids in length, for example when part of a larger fusion protein.

In some examples, an immunogenic Af-associated peptide is an antigen set forth in Table 1. For example, an immunogenic Af-associated peptide is an antigen provided in Table 1, generally between 7 and 20 amino acids in length, such as about 8 to 15 residues in length.

Consecutive repeats of exemplary immunogenic Af-associated peptides can be joined by a peptide linker between two and ten amino acids in length, such as about 2, about 3, about 4, about 5, about 6, about 7, about 8, about 9 or about 10 amino acids in length. Depending on such factors as the molecules to be linked, and the conditions in which the peptide is being administered (such as if the peptide is being used in a method of detection), the linker can vary in length and composition for optimizing such properties as flexibility, and stability. The linker is a peptide heterologous to the immunogenic Af-associated peptide. In some examples, a linker is peptide such as poly-lysine, poly-glutamine, poly-glycine, poly-proline or any combination combinations thereof. In some examples, the peptide linker can be designed to be either hydrophilic or hydrophobic in order to enhance the desired binding characteristics of the immunogenic Af-associated peptide

The peptide linker and the individual units of the immunogenic Af-associated peptide can be encoded as a single fusion polypeptide such that the peptide linker and the individual units of the Af-associated peptide are joined by peptide bonds.

In other embodiments, an immunogenic Af-associated peptide has an amino acid sequence at least 90% identical to a wild-type Af-associated molecule (such as antigen listed in Table 1), for example a polypeptide that has at least about 91%, 92%, 93%, 94%, 95%, 96%, 97%, 98% or 99% sequence identity to a wild-type Af-associated molecule (such as antigen listed in Table 1).

Using the genetic code, one of skill in the art can readily produce a nucleic acid sequence encoding an Af-associated polypeptide (such as an antigen listed in Table 1).

The disclosed polypeptides disclosed herein can be chemically synthesized by standard methods, or can be produced recombinantly. An exemplary process for polypeptide production is described in Lu et al., Federation of European Biochemical Societies Letters. 429:31-35, 1998. They can also be isolated by methods including preparative chromatography and immunological separations.

A disclosed polypeptide can be covalently linked to a carrier, which is an immunogenic macromolecule to which an antigenic molecule can be bound. When bound to a carrier, the bound polypeptide becomes more immunogenic. Carriers are chosen to increase the immunogenicity of the bound molecule and/or to elicit higher titers of antibodies against the carrier which are diagnostically, analytically, and/or therapeutically beneficial. Covalent linking of a molecule to a carrier can confer enhanced immunogenicity and T cell dependence (see Pozsgay et al., PNAS 96:5194-97, 1999; Lee et al., J. Immunol. 116:1711-18, 1976; Dintzis et al., PNAS 73:3671-75, 1976). Useful carriers include polymeric carriers, which can be natural (for example, polysaccharides, polypeptides or proteins from bacteria or viruses), semi-synthetic or synthetic materials containing one or more functional groups to which a reactant moiety can be attached. Bacterial products and viral proteins (such as hepatitis B surface antigen and core antigen) can also be used as carriers, as well as proteins from higher organisms such as keyhole limpet hemocyanin, horseshoe crab hemocyanin, edestin, mammalian serum albumins, and mammalian immunoglobulins. Additional bacterial products for use as carriers include bacterial wall proteins and other products (for example, streptococcal or staphylococcal cell walls and LPS and/or CPS).

ii. Polynucleotides Encoding Immunogenic Aspergillus Species Peptides, Including Af Peptides

Polynucleotides encoding the disclosed polypeptides disclosed herein are also provided. These polynucleotides include DNA, cDNA and RNA sequences which encode the polypeptide of interest. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (e.g., L. Stryer, 1988, Biochemistry, 3.sup.rd Edition, W.H. 5 Freeman and Co., NY).

A nucleic acid encoding an Af-associated polypeptide can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). For example, a polynucleotide encoding a Af protein can be isolated by polymerase chain reaction of cDNA using primers based on the DNA sequence of the molecule. A wide variety of cloning and in vitro amplification methodologies are well known to persons skilled in the art. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, NY, 1989). Polynucleotides also can be isolated by screening genomic or cDNA libraries with probes selected from the sequences of the desired polynucleotide under stringent hybridization conditions.

The polynucleotides encoding an Af-associated polypeptide include a recombinant DNA which is incorporated into a vector in an autonomously replicating plasmid or virus or into the genomic DNA of a prokaryote or eukaryote, or which exists as a separate molecule (such as a cDNA) independent of other sequences. The nucleotides of the disclosure can be ribonucleotides, deoxyribonucleotides, or modified forms of either nucleotide. The term includes single and double forms of DNA.

In one embodiment, vectors are used for expression in yeast such as S. cerevisiae or Kluyveromyces lactis. Several promoters are known to be of use in yeast expression systems such as the constitutive promoters plasma membrane H⁺-ATPase (PMA1), glyceraldehyde-3-phosphate dehydrogenase (GPD), phosphoglycerate kinase-1 (PGK1), alcohol dehydrogenase-1 (ADH1), and pleiotropic drug-resistant pump (PDR5). In addition, may inducible promoters are of use, such as GAL1-10 (induced by galactose), PHO5 (induced by low extracellular inorganic phosphate), and tandem heat shock HSE elements (induced by temperature elevation to 37° C.). Promoters that direct variable expression in response to a titratable inducer include the methionine-responsive MET3 and MET25 promoters and copper-dependent CUP1 promoters. Any of these promoters may be cloned into multicopy (2μ) or single copy (CEN) plasmids to give an additional level of control in expression level. The plasmids can include nutritional markers (such as URA3, ADE3, HIS1, and others) for selection in yeast and antibiotic resistance (AMP) for propagation in bacteria. Plasmids for expression on K. lactis are known, such as pKLAC1. Thus, in one example, after amplification in bacteria, plasmids can be introduced into the corresponding yeast auxotrophs by methods similar to bacterial transformation.

The disclosed peptides can be expressed in a variety of yeast strains. For example, seven pleiotropic drug-resistant transporters, YOR1, SNQ2, PDR5, YCF1, PDR10, PDR11, and PDR15, together with their activating transcription factors, PDR1 and PDR3, have been simultaneously deleted in yeast host cells, rendering the resultant strain sensitive to drugs. Yeast strains with altered lipid composition of the plasma membrane, such as the erg6 mutant defective in ergosterol biosynthesis, can also be utilized. Proteins that are highly sensitive to proteolysis can be expressed in a yeast lacking the master vacuolar endopeptidase Pep4, which controls the activation of other vacuolar hydrolases. Heterologous expression in strains carrying temperature-sensitive (ts) alleles of genes can be employed if the corresponding null mutant is inviable.

Viral vectors can also be prepared encoding the polypeptides disclosed herein. A number of viral vectors have been constructed, including polyoma, SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Nad. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

Thus, in one embodiment, the polynucleotide encoding a disclosed polypeptide is included in a viral vector. Suitable vectors include retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors and poliovirus vectors. Specific exemplary vectors are poxvirus vectors such as vaccinia virus, fowlpox virus and a highly attenuated vaccinia virus (MVA), adenovirus, baculovirus and the like.

Pox viruses useful in practicing the present disclosure can include orthopox, suipox, avipox, and capripox virus. Orthopox includes vaccinia, ectromelia, and raccoon pox. One example of an orthopox of use is vaccinia. Avipox includes fowlpox, canary pox and pigeon pox. Capripox includes goatpox and sheeppox. In one example, the suipox is swinepox. Examples of pox viral vectors for expression are described for example, in U.S. Pat. No. 6,165,460, which is incorporated herein by reference. Other viral vectors that can be used include other DNA viruses such as herpes virus and adenoviruses, and RNA viruses such as retroviruses and polio.

In some cases, vaccinia viral vectors may elicit a strong antibody response. Thus, while numerous boosts with vaccinia vectors are possible, its repeated use may not be useful in certain instances. However, this sensitivity problem can be minimized by using pox from different genera for boosts. In one example, when the first or initial pox virus vector is vaccinia, the second and subsequent pox virus vectors are selected from the pox viruses from a different genus such as suipox, avipox, capripox or an orthopox immunogenically distinct from vaccinia.

The vaccinia virus genome is known in the art. It is composed of a HIND F13L region, TK region, and an HA region. Recombinant vaccinia virus has been used to incorporate an exogenous gene for expression of the exogenous gene product (see, for example, Perkus et al. Science 229:981-984, 1985; Kaufman et al. Int. J. Cancer 48:900-907, 1991; Moss Science 252:1662, 1991). A gene encoding an antigen of interest, such as an immunogenic Af polypeptide, can be incorporated into the HIND F13L region or alternatively incorporated into the TK region of recombinant vaccinia virus vector (or other nonessential regions of the vaccinia virus genome). Baxby and Paoletti (Vaccine 10:8-9, 1992) disclose the construction and use as a vector, of the non-replicating poxvirus, including canarypox virus, fowlpox virus and other avian species. Sutter and Moss (Proc. Nat'l. Acad. Sci U.S.A. 89:10847-10851, 1992) and Sutter et al. (Virology 1994) disclose the construction and use as a vector, the non-replicating recombinant Ankara virus (MVA, modified vaccinia Ankara) in the construction and use of a vector.

Suitable vectors are disclosed, for example, in U.S. Pat. No. 6,998,252, which is incorporated herein by reference. In one example, a recombinant poxvirus, such as a recombinant vaccinia virus is synthetically modified by insertion of a chimeric gene containing vaccinia regulatory sequences or DNA sequences functionally equivalent thereto flanking DNA sequences which in nature are not contiguous with the flanking vaccinia regulatory DNA sequences that encode an Af-associated polypeptide. The recombinant virus containing such a chimeric gene is effective at expressing the Af-associated polypeptide. In one example, the vaccine viral vector comprises (A) a segment comprised of (i) a first DNA sequence encoding a Af polypeptide and (ii) a poxvirus promoter, wherein the poxvirus promoter is adjacent to and exerts transcriptional control over the DNA sequence encoding a Af polypeptide; and, flanking said segment, (B) DNA from a nonessential region of a poxvirus genome. The viral vector can encode a selectable marker. In one example, the poxvirus includes, for example, a thymidine kinase gene (see U.S. Pat. No. 6,998,252, which is incorporated herein by reference).

Poxviral vectors that encode Af-associated polypeptide include at least one expression control element operationally linked to the nucleic acid sequence encoding the Af-associated polypeptide. The expression control elements are inserted in the poxviral vector to control and regulate the expression of the nucleic acid sequence. Examples of expression control elements of use in these vectors includes, but is not limited to, lac system, operator and promoter regions of phage lambda, yeast promoters and promoters derived from polyoma, adenovirus, retrovirus or SV40. Additional operational elements include, but are not limited to, leader sequence, termination codons, polyadenylation signals and any other sequences necessary for the appropriate transcription and subsequent translation of the nucleic acid sequence encoding the Af-associated polypeptide in the host system. The expression vector can contain additional elements necessary for the transfer and subsequent replication of the expression vector containing the nucleic acid sequence in the host system. Examples of such elements include, but are not limited to, origins of replication and selectable markers. It will further be understood by one skilled in the art that such vectors are easily constructed using conventional methods (Ausubel et al., (1987) in “Current Protocols in Molecular Biology,” John Wiley and Sons, New York, N.Y.) and are commercially available.

Basic techniques for preparing recombinant DNA viruses containing a heterologous DNA sequence encoding the Af-associated polypeptide, are known in the art. Such techniques involve, for example, homologous recombination between the viral DNA sequences flanking the DNA sequence in a donor plasmid and homologous sequences present in the parental virus (Mackett et al., 1982, Proc. Natl. Acad. Sci. USA 79:7415-7419). In particular, recombinant viral vectors such as a poxviral vector can be used in delivering the gene. The vector can be constructed for example by steps known in the art, such as steps analogous to the methods for creating synthetic recombinants of the fowlpox virus described in U.S. Pat. No. 5,093,258, which is hereby incorporated by reference. Other techniques include using a unique restriction endonuclease site that is naturally present or artificially inserted in the parental viral vector to insert the heterologous DNA.

Generally, a DNA donor vector contains the following elements: (i) a prokaryotic origin of replication, so that the vector may be amplified in a prokaryotic host; (ii) a gene encoding a marker which allows selection of prokaryotic host cells that contain the vector (e.g., a gene encoding antibiotic resistance); (iii) at least one DNA sequence encoding an Af-associated polypeptide located adjacent to a transcriptional promoter capable of directing the expression of the sequence; and (iv) DNA sequences homologous to the region of the parent virus genome where the foreign gene(s) will be inserted, flanking the construct of element (iii). Methods for constructing donor plasmids for the introduction of multiple foreign genes into pox virus are described in WO91/19803, incorporated herein by reference.

Generally, DNA fragments for construction of the donor vector, including fragments containing transcriptional promoters and fragments containing sequences homologous to the region of the parent virus genome into which foreign DNA sequences are to be inserted, can be obtained from genomic DNA or cloned DNA fragments. The donor plasmids can be mono, di-, or multivalent (i.e., can contain one or more inserted foreign DNA sequences). The donor vector can contain an additional gene that encodes a marker that will allow identification of recombinant viruses containing inserted foreign DNA. Several types of marker genes can be used to permit the identification and isolation of recombinant viruses. These include genes that encode antibiotic or chemical resistance (e.g., see Spyropoulos et al., 1988, J. Virol. 62:1046; Falkner and Moss, 1988, J. Virol. 62:1849; Franke et al., 1985, Mol. Cell. Biol. 5:1918), as well as genes such as the E. coli lacZ gene, that permit identification of recombinant viral plaques by colorimetric assay (Panicali et al., 1986, Gene 47:193-199).

The DNA gene sequence to be inserted into the virus can be placed into a donor plasmid, such as an E. coli or a Salmonella plasmid construct, into which DNA homologous to a section of DNA such as that of the insertion site of the poxvirus where the DNA is to be inserted has been inserted. Separately the DNA gene sequence to be inserted is ligated to a promoter. The promoter-gene linkage is positioned in the plasmid construct so that the promoter-gene linkage is flanked on both ends by DNA homologous to a DNA sequence flanking a region of pox DNA that is the desired insertion region. With a parental pox viral vector, a pox promoter is used. The resulting plasmid construct is then amplified by growth within E. coli bacteria and isolated. Next, the isolated plasmid containing the DNA gene sequence to be inserted is transfected into a cell culture, for example chick embryo fibroblasts, along with the parental virus, for example poxvirus. Recombination between homologous pox DNA in the plasmid and the viral genome respectively results in a recombinant poxvirus modified by the presence of the promoter-gene construct in its genome, at a site that does not affect virus viability.

As noted above, the DNA sequence is inserted into a region (insertion region) in the virus that does not affect virus viability of the resultant recombinant virus. One of skill in the art can readily identify such regions in a virus by, for example, randomly testing segments of virus DNA for regions that allow recombinant formation without seriously affecting virus viability of the recombinant. One region that can readily be used and is present in many viruses is the thymidine kinase (TK) gene. The TK gene has been found in all pox virus genomes examined, including leporipoxvirus (Upton et al., 1986, J. Virology 60:920); shope fibromavirus; capripoxvirus (Gershon et al., 1989, J. Gen. Virol. 70:525) Kenya sheep-1; orthopoxvirus (Weir et al., 1983, J. Virol. 46:530) vaccinia (Esposito et al., 1984, Virology 135:561); monkeypox and variola virus (Hruby et al., 1983, PNAS 80:3411) vaccinia (Kilpatrick et al., 1985, Virology 143:399); Yaba monkey tumor virus; avipoxvirus (Binns et al., 1988, J. Gen. Virol. 69:1275); fowipox; (Boyle et al., 1987, Virology 156:355); fowlpox (Schnitzlein et al., 1988, J. Virological Methods 20:341); fowlpox, quailpox; entomopox (Lytvyn et al., 1992, J. Gen. Virol. 73:3235-3240). In vaccinia, in addition to the TK region, other insertion regions include, for example, the Hindlll M fragment. In fowlpox, in addition to the TK region, other insertion regions include, for example, the BamHI J fragment (Jenkins et al., 1991, AIDS Research and Human Retroviruses 7:991-998) the ECORI-Hindlll fragment, EcoRV-Hindlll fragment, BamHI fragment and the Hindlll fragment set forth in EPO Application No. 0 308220 A1 (see also Calvert et al., 1993, J. Virol. 67:3069-3076; Taylor et al., 1988, Vaccine 6:497-503; Spehner et al., 1990; Boursnell et al., 1990, J. Gen. Virol. 71:621-628).

In swinepox, insertion sites include the thymidine kinase gene region. In addition to the requirement that the gene be inserted into an insertion region, successful expression of the inserted gene by the modified poxvirus requires the presence of a promoter operably linked to the desired gene. Generally, the promoter is placed so that it is located upstream from the gene to be expressed. Promoters are well known in the art and can readily be selected depending on the host and the cell type targeted. In one example, in poxviruses, pox viral promoters are used, such as the vaccinia 7.5K, 40K or fowlpox promoters such as FPV CIA. Enhancer elements can also be used in combination to increase the level of expression. Furthermore, inducible promoters can be utilized.

Homologous recombination between donor plasmid DNA and viral DNA in an infected cell can result in the formation of recombinant viruses that incorporate the desired elements. Appropriate host cells for in vivo recombination are generally eukaryotic cells that can be infected by the virus and transfected by the plasmid vector. Examples of such cells suitable for use with a pox virus are chick embryo fibroblasts, HuTK143 (human) cells, and CV-1 and BSC-40 (both monkey kidney) cells. Infection of cells with pox virus and transfection of these cells with plasmid vectors is accomplished by techniques standard in the art (see U.S. Pat. No. 4,603,112 and PCT Publication No. WO 89/03429).

Following in vivo recombination, recombinant viral progeny can be identified by one of several techniques. For example, if the DNA donor vector is designed to insert foreign genes into the parent virus thymidine kinase (TK) gene, viruses containing integrated DNA will be TK- and can be selected on this basis (Mackett et al., 1982, Proc. Natl. Acad. Sci. USA 79:7415). Alternatively, co-integration of a gene encoding a marker or indicator gene with the foreign gene(s) of interest, as described above, can be used to identify recombinant progeny. One specific non-limiting example of an indicator gene is the E. coli lacZ gene. Recombinant viruses expressing beta-galactosidase can be selected using a chromogenic substrate for the enzyme (Panicali et al., 1986, Gene 47:193). Once a recombinant virus has been identified, a variety of well-known methods can be used to assay the expression of the Aspergillus species-associated sequence encoded by the inserted DNA fragment. These methods include black plaque assay (an in situ enzyme immunoassay performed on viral plaques), Western blot analysis, radioimmunoprecipitation (RIPA), and enzyme immunoassay (EIA).

This disclosure encompasses a recombinant virus comprising more than one antigen of interest for the purpose of having a multivalent vaccine. For example, the recombinant virus may comprise the virus genome or portions thereof, the nucleic acid sequence encoding an Aspergillus species polypeptide, such as an Af polypeptide and a nucleic acid sequence encoding a second antigen of interest.

In one embodiment, a composition is provided that includes a recombinant virus comprising a vaccinia virus genome or portions thereof, the nucleic acid sequence encoding a Af polypeptide and a recombinant virus comprising the nucleic acid sequence encoding the immunostimulatory molecule, B 7.1 alone or in combination with the nucleic acid sequence encoding the immunostimulatory molecule, B7-2, or a recombinant virus containing both the genes for an Af-associated antigen and an immunostimulatory molecule. This disclosure also encompasses a recombinant virus comprising the Af-associated polypeptide that is administered with a second recombinant virus comprising the virus genome or portion thereof, and one or more nucleic acid sequences encoding one or more B7 molecules, such as a recombinant vaccinia virus expressing B7-1 and/or B7-2.

Significant amplification of the immune response against a given antigen generally does not occur without co-stimulation (June et al. (Immunology Today 15:321-331, 1994); Chen et al. (Immunology Today 14:483-486); Townsend et al. (Science 259:368-370)). Freeman et al. (J. Immunol. 143:2714-2722, 1989) report cloning and sequencing of B7-1 gene. Azuma et al. Nature 366:76-79, 1993) report cloning and sequencing B7-2 gene. Thus, in one embodiment the B7-1 gene or the B7-2 genes are administered in conjunction with the Af-associated polypeptide. The insertion of nucleic acids encoding B7-1 and B7-2 into vaccinia virus has been disclosed (see for example, U.S. Pat. No. 6,893,869, incorporated herein by reference; this U.S. patent also discloses the use of a nucleic acid encoding IL-2 in a vaccinia virus). Several vectors including IL-2, B7-1 and B7-2 have been deposited with the American Type Culture Collection (ATCC) on Oct. 3, 1994 under the terms of the Budapest Treaty (for example, rV-CEA/ₙIL-2 (ATCC Designation VR 2480), rV-ₘB7-2 (ATCC Designation VR 2482); and rV-ₘB7-1 (ATCC Designation VR 2483)).

DNA sequences encoding an Aspergillus species-associated polypeptide, such as an Af-associated polypeptide, can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

As noted above, a polynucleotide sequence encoding an Af-associated polypeptide can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

Hosts cells can include microbial, yeast, insect and mammalian host cells. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, Jakoby and Pastan (eds), 1979, Cell Culture. Methods in Enzymology, volume 58, Academic Press, Inc., Harcourt Brace Jovanovich, N.Y.). Examples of commonly used mammalian host cell lines are VERO and HeLa cells, CHO cells, and WI38, BHK, and COS cell lines, although cell lines may be used, such as cells designed to provide higher expression desirable glycosylation patterns, or other features. As discussed above, techniques for the transformation of yeast cells, such as polyethylene glycol transformation, protoplast transformation and gene guns are also known in the art (see Gietz and Woods Methods in Enzymology 350: 87-96, 2002).

Transformation of a host cell with recombinant DNA can be carried out by conventional techniques as are well known to those skilled in the art. Where the host is prokaryotic, such as, but not limited to, E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or virus vectors can be used. Eukaryotic cells can also be co-transformed with polynucleotide sequences encoding an Aspergillus species-associated polypeptide, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982).

### VII. Methods for Treating an Aspergillus Species-Associated Condition

Methods are disclosed for treating a subject having an Aspergillus species-associated condition, such as an Af-associated condition (e.g., aspergillosis, including IA). In some embodiments, these methods include inducing an immune response to an Aspergillus species, such as Af, and/or using an inhibitory nucleic acid, such as a siRNA or antisense molecule, to decrease an Aspergillus species-associated molecule expression in order to treat an Aspergillus species-associated disorder/condition. In some examples, the methods include treating a subject with a disease or condition associated with Af, such as aspergillosis, including IA.

The methods can include selecting a subject in need of treatment, such as a subject that expresses one or more Aspergillus species-associated antigens (such as those provided in Table 1) or exhibits one or more signs or symptoms known to one of skill in the art to be associated with the Aspergillus species. In one example, the method includes administering a therapeutically effective amount of a specific binding agent that preferentially binds to an Aspergillus species-associated molecule, such as an Af-associated molecule. The specific binding agent can be an inhibitor such as a siRNA or an antisense molecule that specifically binds mRNA which translates into a protein associated with Af. Inhibition of an Aspergillus species-associated condition, such as aspergillosis, including IA, does not require 100% inhibition, but can include at least a reduction if not a complete inhibition of cell growth or differentiation associated with a specific pathological condition. Treatment of an Aspergillus species-associated condition by inhibiting or reducing expression of one or more Aspergillus species-associated molecules can include delaying the development of an Af-associated condition, such as IA, in a subject. Treatment of the Aspergillus species-associated condition also includes reducing signs or symptoms associated with the Aspergillus species. In some examples, a decrease or slowing IA progression is an alteration of at least 10%, at least 20%, at least 50%, or at least 75%. In some examples, treatment using the methods disclosed herein is used to prevent reoccurrence of Af or the severity of Af systems if it does reoccur. Treatment can also result in modulation, such as down-regulation, of Af markers (such as one or more antigens listed in Table 1).

Specific binding agents are agents that bind with higher affinity to an Af-associated molecule, than to other molecules. For example, a specific binding agent can be one that binds with high affinity to an Af-associated molecule but does not substantially bind to another gene or gene product. For example, the specific binding agent interferes with gene expression (transcription, processing, translation, post-translational modification), such as, by interfering with a Af-associated mRNA and blocking translation into protein.

A reduction in the expression of an Af-associated protein in a target cell may be obtained by introducing into cells an antisense or other suppressive construct based on the Af-associated molecule coding sequence. For antisense suppression, a nucleotide sequence from an Af-associated molecule encoding sequence, e.g. all or a portion of the Af-associated cDNA or gene, is arranged in reverse orientation relative to the promoter sequence in the transformation vector.

The introduced sequence need not be the full length the gene which encodes the Af-associated protein, and need not be exactly homologous to the equivalent sequence found in the cell type to be transformed. Thus, portions or fragments of a nucleic acid encoding an Af-associated protein could also be used to knock out or suppress expression. Generally, however, where the introduced sequence is of shorter length, a higher degree of identity to the native Af-associated molecule sequence will be needed for effective antisense suppression. The introduced antisense sequence in the vector may be at least 15 nucleotides in length, and improved antisense suppression typically will be observed as the length of the antisense sequence increases. The length of the antisense sequence in the vector advantageously may be greater than 100 nucleotides, and can be up to about the full length of the one or more Af-associated cDNA or gene. For suppression of the Af gene itself, transcription of an antisense construct results in the production of RNA molecules that are the reverse complement of mRNA molecules transcribed from the endogenous Af-associated gene in the cell.

Although the exact mechanism by which antisense RNA molecules interfere with gene expression has not been elucidated, it is believed that antisense RNA molecules bind to the endogenous mRNA molecules and thereby inhibit translation of the endogenous mRNA. Expression of an Af-associated molecule can also be reduced using small inhibitory RNAs, for instance using techniques similar to those described previously (see, e.g., Tuschl et al., Genes Dev 13, 3191-3197, 1999; Caplen et al., Proc. Nat'l Acad. Sci. U.S.A. 98, 9742-9747, 2001; and Elbashir et al., Nature 411, 494-498, 2001). Methods of making siRNA that can be used clinically are known in the art. Exemplary siRNAs are commercially available from several sources, such as Sigma Aldrich and Dharmacon, and therapeutic siRNAs can readily be produced using methods known in the art.

Suppression of endogenous Af-associated molecule expression can also be achieved using ribozymes. Ribozymes are synthetic RNA molecules that possess highly specific endoribonuclease activity. The production and use of ribozymes are disclosed in U.S. Pat. No. 4,987,071 to Cech and U.S. Pat. No. 5,543,508 to Haselhoff. The inclusion of ribozyme sequences within antisense RNAs may be used to confer RNA cleaving activity on the antisense RNA, such that endogenous mRNA molecules that bind to the antisense RNA are cleaved, which in turn leads to an enhanced antisense inhibition of endogenous gene expression.

In certain examples, expression vectors are employed to express the inhibitor nucleic acid, such as the antisense, ribozyme or siRNA molecule (see above for additional information on vectors and expression systems). For example, an expression vector can include a nucleic acid sequence encoding the antisense, ribozyme or siRNA molecule. In a particular example, the vector contains a sequence(s) encoding both strands of a siRNA molecule comprising a duplex. In another example, the vector also contains sequence(s) encoding a single nucleic acid molecule that is self-complementary and thus forms a siRNA molecule. Non-limiting examples of such expression vectors are described in Paul et al., Nature Biotechnology 19:505, 2002; Miyagishi and Taira, Nature Biotechnology 19:497, 2002; Lee et al., Nature Biotechnology 19:500, 2002; and Novina et al., Nature Medicine, online publication Jun. 3, 2003, and additional vectors are described herein.

In other examples, inhibitory nucleic acids, such as siRNA molecules include a delivery vehicle, including inter alia liposomes, for administration to a subject, carriers and diluents and their salts, and can be present in pharmaceutical compositions. Nucleic acid molecules can be administered to cells by a variety of methods known to those of skill in the art, including, but not restricted to, encapsulation in liposomes, by iontophoresis, or by incorporation into other delivery vehicles, such as hydrogels, cyclodextrins, biodegradable nanocapsules, and bioadhesive microspheres, or by proteinaceous vectors (see, for example, O'Hare and Normand, International PCT Publication No. WO 00/53722, see also the additional methods described above).

Alternatively, the nucleic acid/vehicle combination can be locally delivered by direct injection or by use of an infusion pump. Direct injection of the nucleic acid molecules of the disclosure, whether subcutaneous, intramuscular, or intradermal, can take place using standard needle and syringe methodologies, or by needle-free technologies such as those described by Barry et al., International PCT Publication No. WO 99/31262. Other delivery routes include, but are not limited to, oral delivery (such as in tablet or pill form), intrathecal or intraperitoneal delivery (see below). More detailed descriptions of nucleic acid delivery and administration are provided in Sullivan et al., PCT WO 94/02595, Draper et al., PCT Publication No. WO93/23569, Beigelman et al., PCT WO99/05094, and Klimuk et al., PCT Publication No. WO 99/04819, all of which are incorporated by reference herein.

Alternatively, certain siRNA molecules can be expressed within cells from eukaryotic promoters. Those skilled in the art will recognize that any nucleic acid can be expressed in eukaryotic cells using the appropriate DNA/RNA vector (see above). The activity of such nucleic acids can be augmented by their release from the primary transcript by an enzymatic nucleic acid (Draper et al., PCT Publication No. WO 93/23569, and Sullivan et al., PCT Publication No. WO 94/02595).

In other examples, siRNA molecules can be expressed from transcription units (see for example, Couture et al., 1996, TIG 12:510) inserted into DNA or RNA vectors. The recombinant vectors can be DNA plasmids or viral vectors. siRNA expressing viral vectors can be constructed based on, for example, but not limited to, adeno-associated virus, retrovirus, adenovirus, lentivirus or alphavirus. In another example, pol III based constructs are used to express nucleic acid molecules of the invention (see for example, Thompson, U.S. Pat. Nos. 5,902,880 and 6,146,886 and others described above).

The recombinant vectors capable of expressing the siRNA molecules can be delivered as described above, and persist in target cells. Alternatively, viral vectors can be used that provide for transient expression of nucleic acid molecules. Such vectors can be repeatedly administered as necessary. Once expressed, the siRNA molecule interacts with the target mRNA and generates an RNAi response. Delivery of siRNA molecule expressing vectors can be systemic, such as by intravenous or intramuscular administration, by administration to target cells explanted from a subject followed by reintroduction into the subject, or by any other means that would allow for introduction into the desired target cell.

The disclosure is illustrated by the following non-limiting Example.

### EXAMPLE

### In Vivo Microbial Antigen Discovery of Aspergillus Antigens Method

This example provides an in vivo Microbial Antigen Discovery (InMAD) for identification of Aspergillus antigens that are shed into body fluids during infection. Antigens discovered in this manner are targets for immunoassays for diagnosis of invasive aspergillosis or other diseases produced by Aspergillus spp.

### Discovery of Antigen Targets for Immunodiagnosis of Invasive Aspergillosis

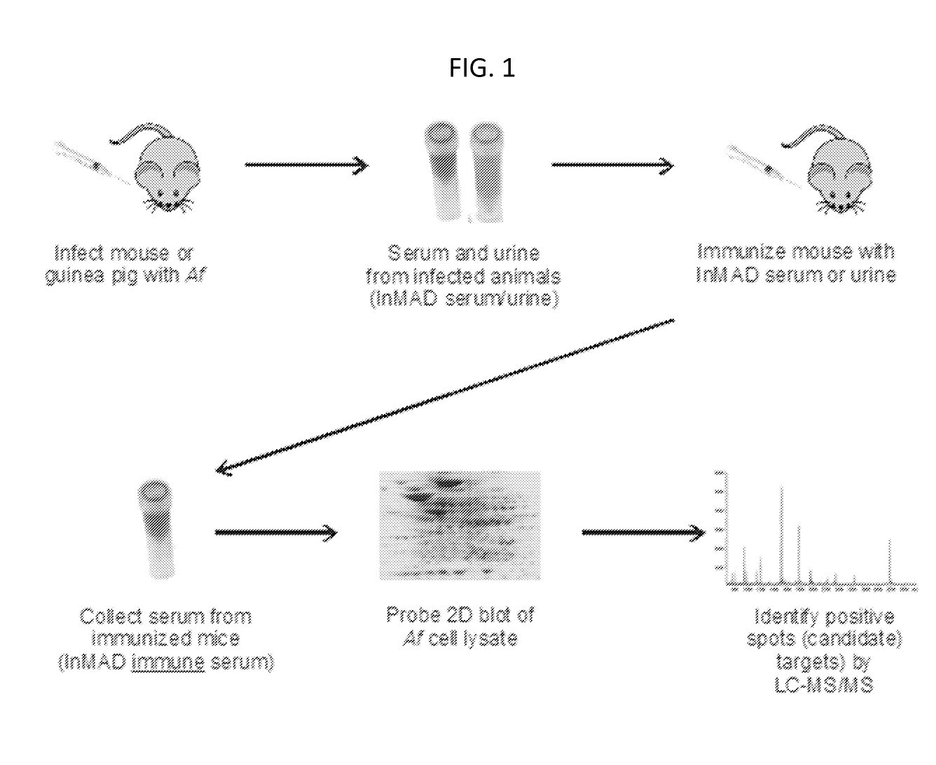

FIG. 1 provides a schematic of InMAD. In the first step, mice, guinea pigs or other host subjects were infected with A. fumigatus. Body fluids, e.g., serum and urine were collected from the infected animals when the animals showed signs of clinical disease. Termed InMAD serum or urine, these samples contain the Aspergillus antigens that are potential targets for diagnosis of aspergillosis. Serum and urine from mice infected with A. fumigatus were obtained from Dr. Scott Filler (Harbor/UCLA Medical Center). Serum and urine from guinea pigs infected with A. fumigatus were obtained from the University of Texas, San Antonio. InMAD serum and urine were filter-sterilized to remove any fungal particles.

In the second step, naïve mice were immunized with InMAD serum or urine. The serum or urine from the infected mice was mixed with TiterMax Gold adjuvant to produce an optimal immune response. Mice were immunized via the subcutaneous route. This immunization step produced antibodies specific for the Aspergillus antigens found in the InMAD serum or urine (termed InMAD immune serum). Presence of antibodies to Aspergillus antigens was determined by Western blots using a lysate of A. fumigatus 293. A. fumigatus 293 was obtained from Dr. Scott Filler (Harbor/UCLA Medical Center). A. fumigatus was grown on RPMI 1640 media plus 2% glucose. The cells were lysed using glass beads and a Mini-Bead Beater. The protein content was determined using EZQ protein assay. Proteins were separated by SDS-PAGE and blotted onto PVDF membranes. Membranes were blocked and then incubated with InMAD immune serum (1:20 dilution). Goat anti-mouse Ig-HRP was used at a 1:10,000 dilution to identify sites for binding of the InMAD immune antibodies. InMAD immune sera that showed reactivity in 1D Western blots with A. fumigatus lysates were pooled for further use.

In the third step, pooled InMAD immune serum was used to probe 2D immunoblots prepared from lysates of A. fumigatus. A. fumigatus protein samples were loaded onto IPG strips. Isoelectric focusing was carried out on a Bio-Rad Protean IEF cell. The strips were layered on gradient gels. Gels were transferred onto nitrocellulose membranes and probed with InMAD immune serum (serum from mice immunized as described above with serum or urine from infected mice or guinea pigs). The total protein pattern on the blots was detected using Sypro Ruby protein stain. A spot cutting set was created using Bio-Rad PDQuest version 8.0 software and spots were then excised using a Bio-Rad ExQuest Spot Cutter.

In the fourth step, mass spectroscopy was performed on spots identified by the immunoblot using a combination ABI 4700 MALDI TOF/TOF and a ThermoElectron LTQ-Orbitrap XL. Selected spots were trypsin digested and the resulting peptides were first separated by a Paradigm Multi-Dimensional Liquid Chromatography (MDLC) instrument (Michrom Bioresources Inc., Auburn Calif.). The Mass spec was operated in data-dependent mode switching between Orbitrap-MS and LTQ-MS/MS. The five most intense data-dependent peaks were subjected to MS/MS using collision-induced dissociation. A reject mass list was used which included known background ions and trypsin fragments. The samples were then searched against the NCBI nr database. Table 1 provides the A fumigatus proteins identified in the immunoblots. Each of these proteins alone or in combination is a target for immunodiagnosis of Aspergillus infection.

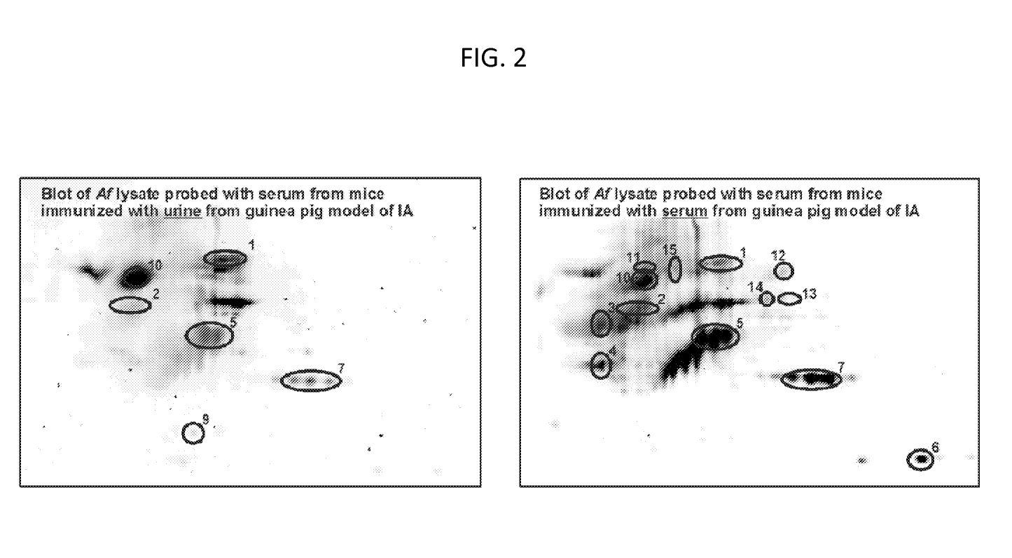

FIG. 2 shows immunoblots in which blots of gels prepared from A. fumigatus lysates were probed with InMAD immune serum from mice immunized with serum or urine from guinea pig models of invasive aspergillosis. The gels illustrate numerous A. fumigatus proteins that were recognized by the InMAD immune serum. Each of these is a potential candidate for a diagnostic test. Fifteen spots were picked and identified by LS-MS/MS. The results of this proteomic analysis are summarized in Table 1. The numbers for each spot shown in FIG. 2 correspond to the numbered proteins in Table 1.

Also listed in Table 1 are Aspergillus proteins identified by use of InMAD serum and urine from neutropenic and non-neutropenic models of invasive aspergillosis to immunize naïve mice and generate further InMAD immune sera. As indicated in Table 1, there was remarkable congruence between Af proteins identified using the various animal models. Similarly, there was congruence between results found using serum and urine from infected animals. These results demonstrate the robustness of the discovery process and the convergence of results onto a limited set of candidate antigens.

TABLE 1
Target discovery via InMAD - Af proteins identified as diagnostic targets by immunization of
mice with serum or urine (InMAD serum and InMAD urine) from i) mouse non-neutropenic,
ii) mouse neutropenic or iii) guinea pig models of IA.

| | | | Mouse Non- | | Mouse | | | |
|---|---|---|---|---|---|---|---|---|
| | | | neutropenic | | Neutropenic | | Guinea pig | |
| No.^{a} | Protein target identified^{b} | Protein ID | Serum | Urine | Serum | Urine | Serum | Urine |
| 1 | Catalase | 70986104 | Y | Y | Y | Y | Y | Y |
| 2 | GPI-anchored cell wall | 70985687 | Y | Y | Y | Y | Y | Y |
| | β-1,3-endogluconase | | | | | | | |
| | (Eglc) | | | | | | | |
| 3 | GPI-anchored cell wall | 70994734 | Y | Y | Y | Y | Y | N |
| | organization protein | | | | | | | |
| | (Ecm33) | | | | | | | |
| 4 | Chr-like protein | 27372089 | Y | N | Y | N | Y | N |
| 5 | Thioreduxin reductase | 70992029 | N | N | Y | N | Y | Y |
| 6 | Peptidyl-prolyl cis-trans | 70989309 | N | N | N | N | Y | Y |
| | isomerase | | | | | | | |
| 7 | Major allergen | 83300352 | N | N | N | N | Y | Y |
| 8 | Conserved hypothetical | 159122886 | Y | N | N | N | N | N |
| | protein | | | | | | | |
| 9 | Conserved hypothetical | 70995516 | Y | N | N | N | N | Y |
| | protein | | | | | | | |
| 10 | 1,3-β- | 70989629 | N | N | N | N | Y | Y |
| | glucanosyltransferase | | | | | | | |
| 11 | Adenoside deaminase | 146323525 | Nd | nd | nd | nd | Y | N |
| 12 | L-amino acid oxidase | 70986680 | Nd | nd | nd | nd | Y | N |
| | Lao | | | | | | | |
| 13 | Glu/Leu/Phe/Val | 70994774 | Nd | nd | nd | nd | Y | N |
| | Dehydrogenase | | | | | | | |
| 14 | Adenosyl | 70995231 | Nd | nd | nd | nd | Y | N |
| | homocysteinase | | | | | | | |
| 15 | Pigment Biosynthesis | 211909651 | Nd | nd | nd | nd | Y | N |
| | Protein | | | | | | | |

^{a}Protein number corresponds to the spots shown in immunoblots of whole cell lysates that were probed with serum from mice immunized with urine or serum from Af-infected guinea pigs (FIG. 2).
^{b}Target proteins were identified by probing 2D blots of whole cell lysates with serum from mice immunized with serum or urine from the indicated animal models of IA. Protein identification was done via proteomics - LC/MS/MS. A “Y” indicates that the protein was identified by proteomic analysis of the immunoblot. An “N”indicates that the protein was not found in the immunoblot. A “nd” indicates that these samples were not used.

### Validation of Candidate Proteins as Potential Candidates for Diagnosis of Aspergillosis

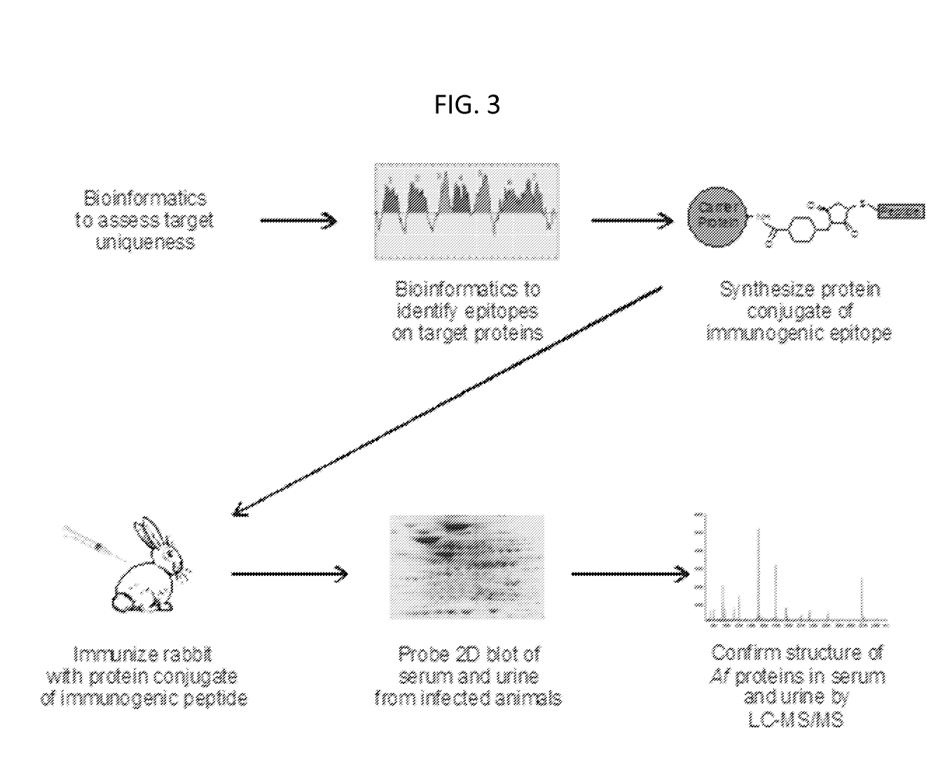

Once potential targets for immunodiagnosis of invasive aspergillosis were identified via the InMAD discovery process, studies were done to validate the proteins as diagnostic targets. The InMAD Target Validation process is described in FIG. 3.

In the first step, five proteins—catalase, GPI-anchored Eglc, GPI-anchored Ecm33, thioreduxin reductase, and chr-like protein, were chosen as lead candidates on the basis of the discovery process that identified the proteins as present in serum and/or urine in multiple models of invasive aspergillosis.

In the second step, a bioinformatics analysis and direct experimentation were done to assess the uniqueness of each protein. That is, is a protein with the same sequence produced by multiple species of Aspergillus and not produced by mammalian cells? The amino acid sequences of the first 10 candidate diagnostic targets were queried via UNIPROTKB/BLAST for homology to other microbial and host proteins. There is some homology across the phyla and kingdoms. However, there is a clear hierarchy of homology for each protein. The results and interpretations are summarized in Table 2.

TABLE 2
Bioinformatics analysis - homology of candidate targets across species,
genera, phyla and kingdoms.

There was a clear hierarchy of homology of target proteins across the
genera, phyla and kingdoms, with Aspergillus spp. >>> other
disease-producing fungi >>>> Bacteria >>>> Mammalian
cells when the total protein sequences were compared.
Overall, there was very strong homology for the proteins across
Aspergillus spp., suggesting that a single immunoassay can be
constructed that will target all of the major Aspergillus spp. that
produce IA.
Regardless of the genus, phylum or kingdom, there were various
amounts of overall protein homology. This is not surprising. Issues of
potential homology overlap are easily addressed by selecting non-
homologous sequences for antibody production.

In a related study, the InMAD immune serum from mice immunized with urine from the guinea pig model of IA was also used to probe whole cell lysates of different Aspergillus spp. and other fungi that are causes of invasive fungal disease in patients who are at high risk for invasive aspergillosis. The goal was to assess the extent of reactivity and cross-reactivity of the InMAD immune serum across Aspergillus spp. and with other medically relevant fungi. These results are summarized in Table 3.

TABLE 3
Reactivity of InMAD immune serum (serum from mice immunized with
urine from guinea pig model of IA) with whole cell lysates of
Aspergillus spp. and other fungi.

2D-blots of cell lysates were probed with pooled serum from mice
immunized with urine from a guinea pig model of IA.
Reactivity of serum was evaluated with lysates of A. fumigatus, A. flavus,
A. niger, A. terreus, and A. nidulans.
No single diagnostic target was identified in every Aspergillus spp.
However, a combination of targets correctly identified every Aspergillus
spp.
There was no reactivity of the InMAD immune serum with 9 of the first
10 potential diagnostic targets (see Table 1) in lysates of Mucor,
Rhizopus and Fusarium, opportunistic fungi that might
cause disease in patients at risk for IA.
The presence of the target proteins across Aspergillus spp. and the
absence of the proteins in Mucor, Rhizopus and Fusarium were
confirmed by LC-MS/MS.

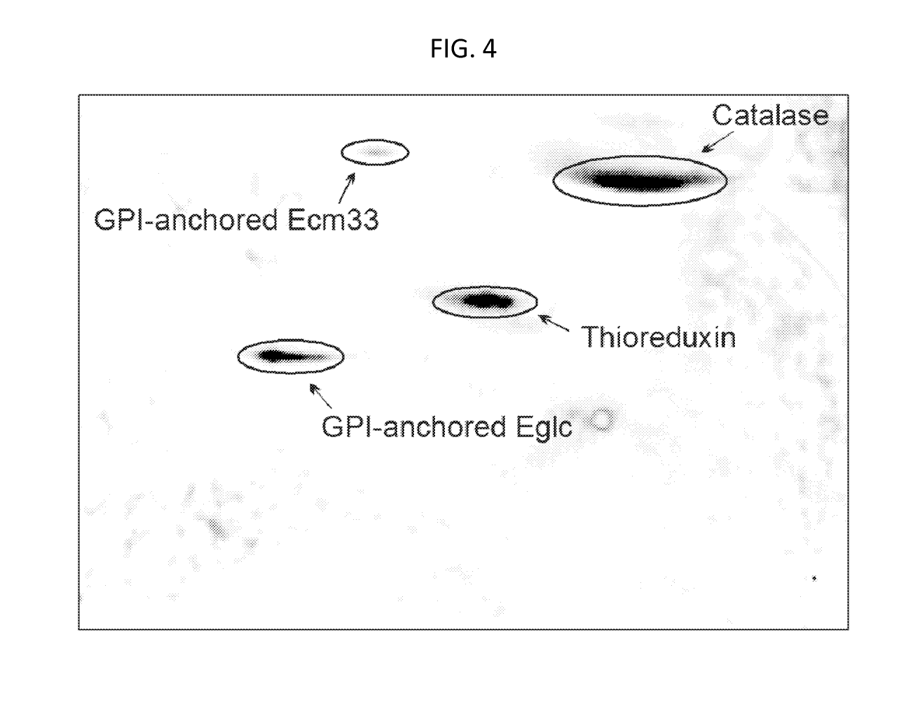

In the third validation step, a bioinformatics analysis was done to identify immunogenic peptides of several proteins shown in Table 1. The peptides were synthesized and coupled to an immunogenic protein carrier. Rabbits were immunized with the peptide-carrier conjugates. Sera were collected from the immunized rabbits and peptide-specific antibodies were purified by immuno-affinity purification. The affinity-purified antibodies to peptides from each protein were then used to probe 2D immunoblots prepared from urine of infected guinea pigs. By way of illustration, a 2D immunoblot from urine that had been probed by a pool of antibodies specific for catalase, GPI-anchored Ecm33, GPI-anchored Eglc and Thioreduxin is shown in FIG. 4. The blot identified proteins that were in positions suggestive of the expected proteins.

In the fourth validation step, the identities of the proteins in the four spots (catalase, GPI-anchored Ecm33, GPI-anchored Eglc and thioreduxin) were determined by mass spectrometry. In all four cases, the proteins in urine were identified as the expected Af proteins, i.e., catalase, GPI-anchored Ecm33, GPI-anchored Eglc and thioreduxin. These results close the loop on target discovery and validation—from target discovery using the InMAD approach→production of antibodies to candidate targets→confirmation that the targets are in serum and urine from infected animals→final verification of the identity of the proteins in samples from the animal models.

The study illustrated in FIG. 4 has been repeated using serum from the guinea pig model of invasive aspergillosis and pooled urine from a second guinea pig challenge model of invasive aspergillosis. The results were identical; all four proteins were confirmed as being i) present in serum and urine from the guinea pig model of invasive aspergillosis and ii) being recognized by antibodies raised against putative antigenic peptides of each target protein, i.e., catalase, GPI-anchored Ecm33, GPI-anchored Eglc and thioreduxin.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method of diagnosing a subject with an Aspergillus species-associated condition or monitoring the efficacy of a therapy, comprising:
detecting at least one Aspergillus species-associated molecule in a sample obtained from a subject exhibiting one or more signs or symptoms associated with an Aspergillus species-associated condition or a subject known to be at risk of acquiring an Aspergillus species-associated condition, wherein the at least one Aspergillus species-associated molecule is at least one antigen listed in Table 1, thereby diagnosing the subject with the Aspergillus species-associated condition or determining the efficacy of the therapy.

2. The method of claim 1, wherein the Aspergillus species is Aspergillus fumigatus.

3. The method of claim 1, further comprising comparing detection of the at least one Aspergillus species-associated molecule in the sample obtained from the subject exhibiting one or more signs or symptoms associated with the Aspergillus species-associated condition to a control, wherein increased detection of the at least one Aspergillus species-associated molecule relative to a control indicates that the subject has the Aspergillus species-associated condition.

4. The method of claim 1, wherein detecting of the at least one Aspergillus species-associated molecule comprises usage of at least one antibody specific for the at least one Aspergillus species-associated molecule.

5. The method of claim 1, wherein the method is used for diagnosing a subject with aspergillosis, such as invasive aspergillosis.

6. (canceled)

7. The method of claim 1, wherein detecting at least one Aspergillus species-associated molecule comprises using an ELISA or a lateral flow device.

8. (canceled)

9. The method of claim 1, wherein the sample is a urine sample, a serum sample, a whole blood sample, or a bronchoalveolar lavage fluid sample.

10.-12. (canceled)

13. A method of monitoring an Aspergillus species-associated condition, comprising:
detecting at least one Aspergillus species-associated molecule in a sample obtained from a subject exhibiting one or more signs or symptoms associated with an Aspergillus species-associated condition or a subject known to be at risk of acquiring an Aspergillus species-associated condition, wherein the at least one Aspergillus species-associated molecule is at least one antigen listed in Table 1, thereby monitoring the Aspergillus species-associated condition.

14. The method of claim 13, wherein the method is used to monitor the efficacy of a therapy to treat the Aspergillus species-associated condition in a subject.

15. The method of claim 14, further comprising comparing detection of the at least one Aspergillus species-associated molecule in the sample obtained from the subject exhibiting one or more signs or symptoms associated with the Aspergillus species-associated condition to a control, wherein decreased detection of the at least one Aspergillus species-associated molecule relative to a control indicates that the treatment of the Aspergillus species-associated condition is effective.

16. The method of claim 13, wherein the method is used to monitor for reoccurrence.

17. The method of claim 16, further comprising comparing detection of the at least one Aspergillus species-associated molecule in the sample obtained from the subject known to be at risk of acquiring an Aspergillus species-associated condition, wherein increased detection of the at least one Aspergillus species-associated molecule relative to a control indicates that the Aspergillus species-associated condition has reoccurred and treatment needs to resume.

18. The method of claim 1, wherein detecting of the at least one Aspergillus species-associated molecule comprises usage of at least one antibody specific for the at least one Aspergillus species-associated molecule.

19. The method of claim 1, wherein the Aspergillus species is Aspergillus fumigatus.

20. The method of claim 1, wherein the method is used to monitor aspergillosis.

21. The method of claim 1, wherein detecting at least one Aspergillus species-associated molecule comprises using an ELISA or a lateral flow device.

22. (canceled)

23. The method of claim 13, wherein the sample is a urine sample, a serum sample, a whole blood sample, or a bronchoalveolar lavage fluid sample.

24.-26. (canceled)

27. A kit for detecting an Aspergillus species-associated condition, comprising at least one molecule capable of detecting at least one Aspergillus species-associated molecule presented in Table 1 and directions for using the kit to detect an Aspergillus species-associated condition.

28. (canceled)

29. (canceled)

30. The kit of claim 27, wherein the kit is one for self monitoring and the at least one molecule capable of detecting at least one Aspergillus species-associated molecule presented in Table 1 is presented on a test strip, such as a dipstick test strip.

31. The kit of claim 27, wherein the Aspergillus species is Aspergillus fumigatus.

32. (canceled)
